(19) United States
(12) Patent Application Publication
(10) Pub. No.: US 2017/0281158 A1
(43) Pub. Date: Oct. 5, 2017

(54) SURGICAL STAPLES

(71) Applicants: William Lear, Corvallis, OR (US); Jennifer Akeroyd, Corvallis, OR (US)

(72) Inventors: William Lear, Corvallis, OR (US); Jennifer Akeroyd, Corvallis, OR (US)

(21) Appl. No.: 15/472,816

(22) Filed: Mar. 29, 2017

Related U.S. Application Data
(60) Provisional application No. 62/318,030, filed on Apr. 4, 2016.

(51) Int. Cl. A61B 17/064; A61B 17/068; A61B 17/072

(52) U.S. Cl. CPC A61B 17/0644; A61B 17/072; A61B 17/0684; A61B 2017/07278

(57) ABSTRACT

A staple is disclosed. The staple includes a crown extending substantially along an axis between a first end and a second end, a first leg extending from the first end of the crown, the first leg ending in a point, and a second leg extending from the second end of the crown, the second leg ending in a point. The first leg and the second leg are deformably moveable between a first configuration and a second configuration. In the first configuration, the first leg and the second leg are positioned substantially parallel with the crown. In the second configuration the first leg and the second leg positioned are substantially perpendicular to the crown. The staple also includes a pad disposed on at least a portion of the crown.

### CROSS REFERENCE TO RELATED APPLICATIONS

This application claims priority to copending U.S. application Ser. No. 62/318,030, filed on Apr. 4, 2016, which is hereby incorporated by reference for all purposes.

### BACKGROUND

### Field

The present disclosure relates to devices, systems, and methods for closing an incision, excision, surgical cut, laceration, or other opening in the patient's skin, herein referred to an incision or wound. In particular, this disclosure describes and relates to staples.

### Description

Surgical staples are one of many different methods used to close wounds or incisions. The staple holds the wound together as the wound heals. However, surgical staples that contact the surface of the user's skin can be inflammatory and/or become ingrown, and can actually impede healing of the wound or incision. Accordingly, in many cases, surgical staples may need to be removed before they become ingrown or infected, which may leave the healing wound or incision in a weakened state.

### SUMMARY

In a first aspect, a staple is disclosed. The staple includes a crown extending substantially along an axis between a first end and a second end, a first leg extending from the first end of the crown, the first leg ending in a point, and a second leg extending from the second end of the crown, the second leg ending in a point. The first leg and the second leg are deformably moveable between a first configuration and a second configuration. In the first configuration, the first leg and the second leg are positioned substantially parallel with the crown. In the second configuration, the first leg and the second leg are positioned substantially perpendicular to the crown. The staple also includes a pad disposed on at least a portion of the crown.

The pad may surround at least a portion of the crown. The pad may be positioned substantially at the midpoint of the crown between the first end and the second end. The pad may comprise a substantially cylindroid-shaped body.

The pad may comprise a substantially rectangular body, and the crown may extend through the rectangular body from a first side of the rectangular body to a second side of a rectangular body. The first side and/or the second side of the rectangular body may include a groove formed therein. The groove may extend along an axis substantially perpendicular to the crown. A top surface of the rectangular body may include a notch formed therein. The notch may extend along an axis substantially perpendicular to the crown. A bottom surface of the rectangular body may include an eversion recess formed therein. The eversion recess may extend through the rectangular body along an axis substantially perpendicular to the crown. The staple may comprise a foot positioned on each side of the eversion recess.

The pad may comprise silicone overmolded on the crown. The pad may comprise polypropylene overmolded on the crown. The pad may comprise any synthetic polymer such as silicone, TPE, TPU, polypropylene, or nylon. The pad may comprise any soft synthetic polymer of type A shore durometer of 110 or less. The pad may comprise any hard synthetic polymer of type D shore durometer of 0 or more. The pad may comprise a first pad and the staple may also comprise a second pad disposed on at least another portion of the crown. The first pad may be separated from the second pad. The first pad and the second pad may each comprise a substantially cylindroid shape. The first pad and the second pad may each comprise silicone overmolded on the crown. The pad may be configured to elevate the crown above a surface of a patient's skin when the staple is in use.

In some embodiments of the staple, each of the first leg and the second legs comprise an extension portion extending from the crown, and a tooth extending from the end of the extension portion to a distal end, the point located at the distal end of the tooth. The tooth may extend substantially orthogonally from the extension portion. The tooth may be curved.

In another aspect, another staple is disclosed. The staple includes a crown extending between a first end and a second end, a first leg extending from the first end of the crown, the first leg ending in a point, and a second leg extending from the second end of the crown, the second leg ending in a point. The first leg and second leg are deformably moveable between a first configuration and a second configuration. In the first configuration, the first leg and the second leg are positioned substantially parallel with the crown. In the second configuration, the first leg and the second leg are positioned substantially perpendicular to the crown. The crown is shaped such that at least a portion of the crown is elevated above a surface of a patient's skin when the staple is in use.

The crown may comprise a central portion located between the first end and the second end, the central portion comprising a U-shape. The U-shape may extend in the same direction as the first leg and the second leg in the second configuration. The U-shape may extend in the opposite direction as the first leg and the second leg in the second configuration. The staple may also include a pad disposed on a bottom portion of the U-shape. The pad may surround at least a portion of the crown. The pad may comprise a substantially cylindroid shape. The pad may comprise an H-shape, and the central portion of the U-shape may extend through a central portion of the H-Shape. In the first configuration, lateral portions of the H-shape may be substantially parallel with the first leg and the second leg. In the second configuration, the first leg and the second leg may be perpendicular to and extend between the lateral portions of the H-shape. The pad may comprise silicone overmolded on the crown.

The crown may comprise a first U-shape adjacent to the first end and second U-shape adjacent to the second end. The first U-shape and the second U-shape may be located in a single plane. The crown may also comprise a central portion located between the first U-shape and the second U-shape. The staple may also include a pad disposed on at least a portion of the central portion. The pad may surround at least a portion of the crown. The pad may comprise a substantially cylindroid shape. The pad may comprise silicone overmolded on the crown.

In another aspect, a method for closing an incision is disclosed. The method comprises positioning a pressure relief device proximal to a wound. The pressure relief device may include a body and at least one feature formed in the body, the feature configured in size and shape to receive a portion of a staple or a suture and elevate the portion above the a surface of a patient's skin when the device is in use. The method also comprises closing the incision with a staple or a suture, at least a portion of the staple or suture formed over pressure relief device and positioned in the feature.

Said positioning step may further comprise positioning the pressure relief device alongside the incision. Said positioning step may further comprise positioning the pressure relief device across the incision. Said closing step may comprise forming a simple interrupted suture, a horizontal mattress suture, or a vertical mattress suture. Said closing step may further comprise forming a knot of the simple interrupted suture, the horizontal mattress suture, or the vertical mattress suture on a top surface of the body. Said closing step may comprise using a staple and a suture. The feature may comprise at least one of a groove formed in a top surface of the body, an indentation formed in a side surface of the body, or an opening extending through the body, among others.

In another aspect, a pressure relief device is disclosed. The pressure relief device comprises a body including a central portion having a first end and second end opposite the first end. The body also includes a first pair of legs extending from the first end, the legs spaced apart to form a slot therebetween, and a second pair of legs extending from the second end, the legs spaced apart to form a slot therebetween. The device may further comprise an eversion recess formed in a bottom surface of the body.

In another aspect, another pressure relief device is disclosed. The device includes a plurality of bodies arranged along an axis. Each body may include a central portion having a first end and second end opposite the first end and a first side and a second side opposite the first side, a first pair of legs extending from the first end, the legs spaced apart to form a slot therebetween, and a second pair of legs extending from the second end, the legs spaced apart to form a slot therebetween. The device also includes a plurality of pairs of bridges extending between adjacent bodies. Each pair of bridges extends between the second side of one body and the first side of the next body. The device may further include an eversion recess formed in a bottom surface of each of plurality the bodies. Each of the bridges may comprise a substantially cylindrical or substantially rectangular shape, among other shapes. In some embodiments, each pair of bridges may be replaced by a single bridge.

In another aspect, a pressure relief device is disclosed. The device includes a body comprising a central portion having a first end and second end opposite the first end. The body also includes a first flange extending from the first end. The first flange includes a first groove formed on one side thereof and a second groove formed on an opposite side thereof. The body also includes a second flange extending from the second end. The second flange includes a first groove formed on one side thereof and a second groove formed on an opposite side thereof. The device may further comprise an eversion recess formed in a bottom surface of the body.

In another aspect, another pressure relief device is disclosed. The device includes a plurality of bodies arranged along an axis. Each body includes a central portion having a first end and second end opposite the first end and a first side and a second side opposite the first side. Each body includes a first flange extending from the first end, the first flange including a first groove formed on one side thereof and a second groove formed on an opposite side thereof. Each body includes a second flange extending from the first end, the second flange including a first groove formed on one side thereof and a second groove formed on an opposite side thereof. The device also includes a plurality of pairs of bridges extending between adjacent bodies. Each pair of bridges extends between the second side of one body and the first side of the next body. The device may further include an eversion recess formed in a bottom surface of each of plurality the bodies. Each of the bridges may comprise a substantially cylindrical or substantially rectangular shape, among other shapes. In some embodiments, each pair of bridges may be replaced by a single bridge.

### BRIEF DESCRIPTION OF THE DRAWINGS

The features and advantages of the devices and methods described herein will become more fully apparent from the following description and appended claims, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1A shows an embodiment of a surgical staple in a first configuration and positioned above an incision.

FIG. 1B shows the surgical staple of FIG. 1A in a second configuration, closing an incision.

FIG. 2A shows a perspective view of one embodiment of staple remover tool removing a surgical staple.

FIGS. 2B-2E show views illustrating how a surgical staple is removed using the tool of FIG. 2A.

FIG. 3A shows a perspective view of one embodiment of a surgical staple including a pad, illustrated in a first configuration.

FIG. 3B shows the surgical staple of FIG. 3A illustrated in a second configuration.

FIG. 4A shows a perspective view of one embodiment of a surgical staple including two pads, illustrated in a first configuration.

FIG. 4B shows the surgical staple of FIG. 4A illustrated in a second configuration.

FIG. 5A shows a perspective view of another embodiment of a surgical staple including a pad, illustrated in a first configuration.

FIG. 5B shows the surgical staple of FIG. 5A illustrated in a second configuration.

FIGS. 5C and 5D show front views illustrating how the surgical staple of FIG. 5A may be removed in some embodiments.

FIG. 6A shows a perspective view of another embodiment of a surgical staple including a pad, illustrated in a first configuration.

FIG. 6B shows the surgical staple of FIG. 6A illustrated in a second configuration.

FIG. 7A shows a perspective view an embodiment of a surgical staple, in a first configuration, with a crown shaped so that a portion of the crown is elevated above a surface of the skin when in use. The surgical staple also includes a pad.

FIG. 7B shows the surgical staple of FIG. 7A illustrated in a second configuration.

FIG. 8A shows a perspective view of another embodiment of surgical staple, in a first configuration, with a crown shaped so that a portion of the crown is elevated above a surface of the skin when in use. The surgical staple also includes an H-shaped pad.

FIG. 8B shows the surgical staple of FIG. 8A illustrated in a second configuration.

FIG. 9A shows a perspective view another embodiment of a surgical staple, in a first configuration, with a crown shaped so that a portion of the crown is elevated above a surface of the skin when in use.

FIG. 9B shows the surgical staple of FIG. 9A illustrated in a second configuration.

FIG. 10A shows a perspective view another embodiment of the surgical staple of FIG. 9A with a pad, illustrated in a first configuration.

FIG. 10B shows the surgical staple of FIG. 10A, illustrated in a second configuration.

FIG. 11A shows a perspective view of another embodiment of a surgical staple, in a first configuration, with a crown shaped so that a portion of the crown is elevated above a surface of the skin when in use.

FIG. 11B shows the surgical staple of FIG. 11A in a second configuration.

FIG. 11C shows a perspective view of another embodiment of a surgical staple, in a first configuration, with a crown shaped so that a portion of the crown is elevated above a surface of the skin when in use.

FIG. 11D shows the surgical staple of FIG. 11A in a second configuration where the portion of the crown is elevated above the surface of the skin when in use.

FIG. 12A shows a perspective view another embodiment of the surgical staple of FIG. 11A with a pad, illustrated in a first configuration.

FIG. 12B shows the surgical staple of FIG. 12A, illustrated in a second configuration.

FIG. 13A shows a side view of one embodiment of a pressure relief device.

FIG. 13B shows a top view of the pressure relief device of FIG. 13A.

FIG. 13C shows a cross-sectional view of the pressure relief device of FIG. 13A taken through a surgical staple.

FIG. 13D shows a cross-sectional view of the pressure relief device of FIG. 13B taken through a suture.

FIG. 14A shows a perspective view of another embodiment of a pressure relief device.

FIG. 14B shows the pressure relief device of FIG. 14A in use with a simple interrupted suture.

FIG. 14C shows an embodiment of a pressure relief device configured for use with multiple sutures.

FIG. 14D shows another embodiment of a pressure relief device configured for use with multiple sutures.

FIG. 15A shows a perspective view of another embodiment of a pressure relief device.

FIG. 15B shows the pressure relief device of FIG. 15A in use with a horizontal mattress suture.

FIG. 15C shows another embodiment of a pressure relief device configured for use with multiple sutures.

FIG. 16A shows a perspective view of another embodiment of a pressure relief device.

FIG. 16B shows the pressure relief device of FIG. 16A in use with a simple interrupted suture.

FIG. 16C shows another embodiment of a pressure relief device configured for use with multiple sutures.

### DETAILED DESCRIPTION

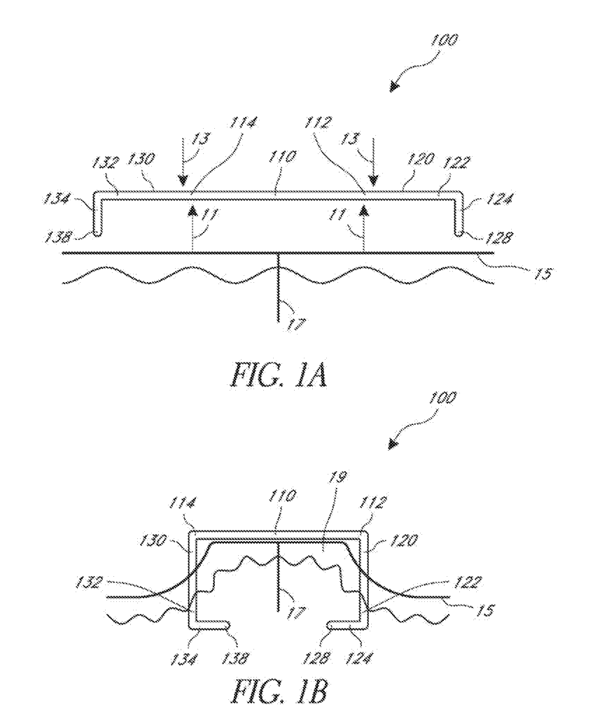

FIG. 1A shows an embodiment of a surgical staple 100 in a first configuration and positioned above an incision 17 in a patient's skin 15. The incision 17 may be a surgical cut, excision, laceration, or other opening in the patient's skin 15, herein referred to an incision or wound.

The staple 100 may be used to close the incision 17. The staple 100 includes a crown 112, a first leg 120, and a second leg 130. The crown 112 extends between a first end 112 and a second end 114. In the illustrated embodiment, the crown extends substantially linearly, or along a substantially straight axis, between the first end 112 and the second end 114. The first leg 120 extends from the first end 112 of the crown 110. The second leg 130 extends from the second end 114 of the crown 110. In some embodiments, the crown 110, the first leg 120, and the second leg 130 are all integrally formed. The first leg 120 may end in a point 128. The second leg 130 may end in a point 138. The points 128, 138 may be tissue piercing points, configured to pierce the patient's skin 15.

In the illustrated embodiment, the first leg 120 includes an extension portion 122 that extends away from the first end 112 of the crown 110. A distal end of the extension portion 122 is connected to a tooth 124. In the illustrated embodiment, the tooth 124 extends substantially orthogonally from the extension portion 124. The point 128 may be positioned at a distal end of the tooth 124. Similarly, the second leg 130 may include an extension portion 132 that extends away from the second end 114 of the crown 110. A distal end of the extension portion 132 is connected to a tooth 134. In the illustrated embodiment, the tooth 134 extends substantially orthogonally from the extension portion 134. The point 138 may be positioned at a distal end of the tooth 134. In general, the teeth 124, 134 may extend in substantially the same direction. For example, in FIG. 1A, the teeth 124, 134 each extend in a downward direction relative to the orientation shown in the figure. In some embodiments, the teeth 124, 134 may be curved relative to the extension portions 122, 134.

In some embodiments, the staple 100 may include an integrally-formed structure. For example, the staple 100 may include a single piece of metal, for example, a surgical steel, or other suitable material, bent into the configuration illustrated in FIG. 1A. In some embodiments, the cross-section of the staple 100 is substantially constant. In some embodiments, the cross-section is substantially annular, rectangular, or any other shape.

In FIG. 1A, the staple 100 is illustrated in a first configuration. The first configuration may represent an initial configuration of the staple 100, such as, the configuration in which the staple 100 is manufactured or the configuration in which the staple 100 is placed in a stapler before stapling. In the first configuration, the crown 110, the extension portion 122 of the first leg 120, and the extension portion 132 of the second leg 130 may all be oriented to be parallel to each other. In some embodiments, the crown 110, the extension portion 122 of the first leg 120, and the extension portion 132 of the second leg 130 are all positioned substantially along a single axis. In some embodiments, at least the first end 112 and the second end of 114 of the crown 110 may be deformable, such that the staple 100 can be transitioned to a second configuration, illustrated in FIG. 1B.

Arrows 11, 13 in FIG. 1A illustrate an example of how force may be applied to the staple 100, for example by a stapler, to transition the staple 100 from the first configuration to the second configuration. Arrows 11 represent a stationary upward force that may be applied just inside (toward the center of the crown 110) the first end 112 and the second end 114. This force may be applied by an anvil of a stapler. Arrows 13 represent a moving force 13 that may be applied just outside (away from the center of the crown 110) the first end and the second 114. This force may be applied by a hammer of the stapler. Because the arrows 11 represent a stationary force and the arrows 13 represent a moving force, the forces cause the first arm 120 and the second arm 130 of the staple 100 to bend relative to the crown 110. The first arm 120 and the second arm 130 may bend relative to the crown into the second configuration illustrated in FIG. 1B.

FIG. 1B shows the surgical staple 100 of FIG. 1A in the second configuration. The second configuration may represent a deformed configuration, such as a stapled or installed configuration. In general, the staple 100 is used in the second configuration to close the incision 17. In the second configuration, the extension portion 122 of the first leg 120 is bent relative to the crown 110. In some embodiments, the angle between the crown 110 and the extension portion 122 of the first leg 120 may be approximately 90 degrees, although greater or smaller angles may be used in other embodiments. Similarly, in the second configuration, the extension portion 132 of the second leg 130 is bent relative to the crown 110. In some embodiments, the angle between the crown 110 and the extension portion 132 of the second leg 130 may be approximately 90 degrees, although greater or smaller angles may be used in other embodiments. In some embodiments, the first leg 120 and the second leg 130 are each bent to point in substantially the same direction. For example, in FIG. 1B, the first leg 120 and the second leg 130 each point substantially downward relative to the orientation of the figure. In some embodiments, in the second configuration, the crown 110, the extension portion 122 of first leg 120, and the extension portion 132 of the second leg 130 are all located in a single plane. In some embodiments, the teeth 124, 134 are also located in the same plane.

In some embodiments, when the staple 100 is used to close the incision 17, the staple creates a region of wound eversion 19 around the incision 17, as shown in FIG. 1B. The region of wound eversion 19 may be a raised portion of the patient's tissue and skin relative to the surface of the skin 15. In some embodiments, the crown 110 rests on top of the skin 15 and the region of wound eversion 19. As the staple 100 transitions from the first configuration (FIG. 1A) to the second configuration (FIG. 1B), the points 128, 138 pierce the skin 15. The teeth 124, 134 enter the patient's tissue, and as the arms 120, 130 rotate downward, the tissue may be gathered together to form the region of wound eversion 19. In the illustrated embodiment, in the second configuration the teeth 124, 134 are positioned substantially parallel to the crown 110. The teeth 124, 134 may thus anchor the staple 100 in place.

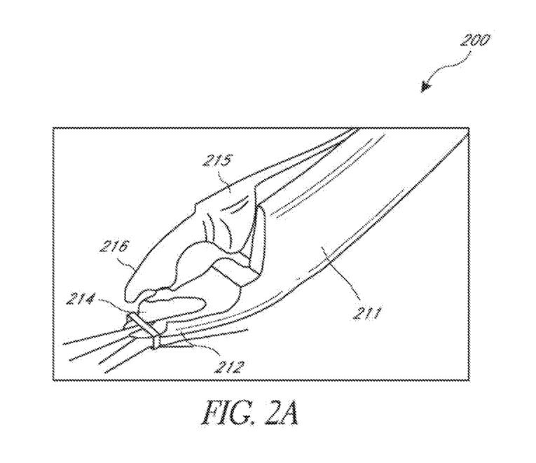

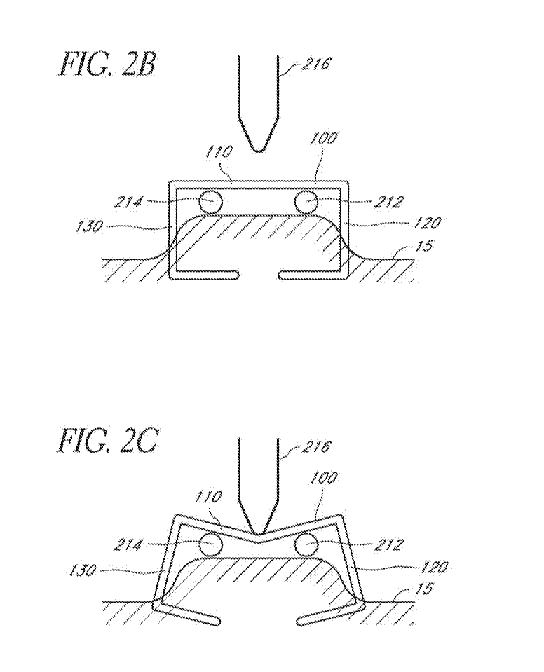

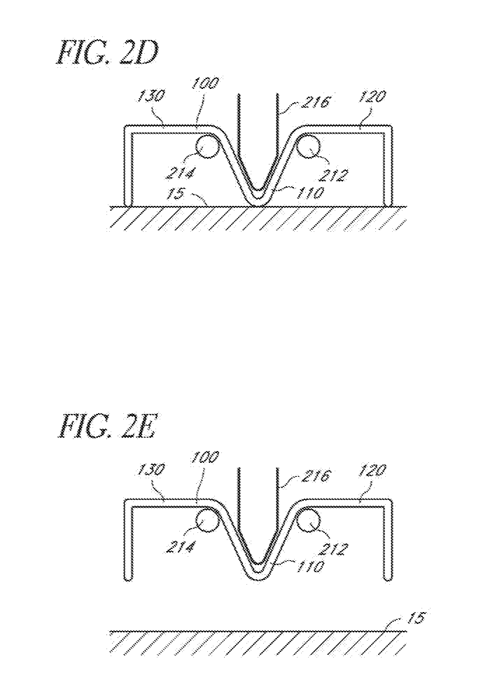

FIG. 2A shows a perspective view of one embodiment of staple remover tool 200 removing the surgical staple 100. The tool 200 includes a first arm 211 and a second arm 215 that rotate relative to each other in a scissor-like manner. The first arm 211 terminates in two prongs 212, 214 spaced apart from each other. The second arm 215 terminates in a single prong 216. The single prong 216 is positioned centrally between the two prongs 212, 214. FIGS. 2B-2E show views illustrating how the surgical staple 100 is removed using the tool 200 of FIG. 2A. As shown in FIG. 2B, the two prongs 212, 214 are worked between the skin 15 and the crown 110. In FIG. 2B, the single prong 216 is rotated downward to contact the crown 110. As the single prong 212 is rotated below an axis extending between the two prongs 212, 214, the crown 110 begins to deform causing the first arm 120 and the second arm 130 of the staple 100 to rotate into an open position. As shown in FIG. 2C, the single prong 216 is rotated downward until the first arm 120 and the second arm 130 of the staple 130 are free from the skin 15. As shown in FIG. 2D, the staple 100 may then be removed. The tool 200 and the process for removal of the staple 100 illustrated in FIGS. 2A-2E are provided by way of example only. Other tools and processes for removing a staple are possible.

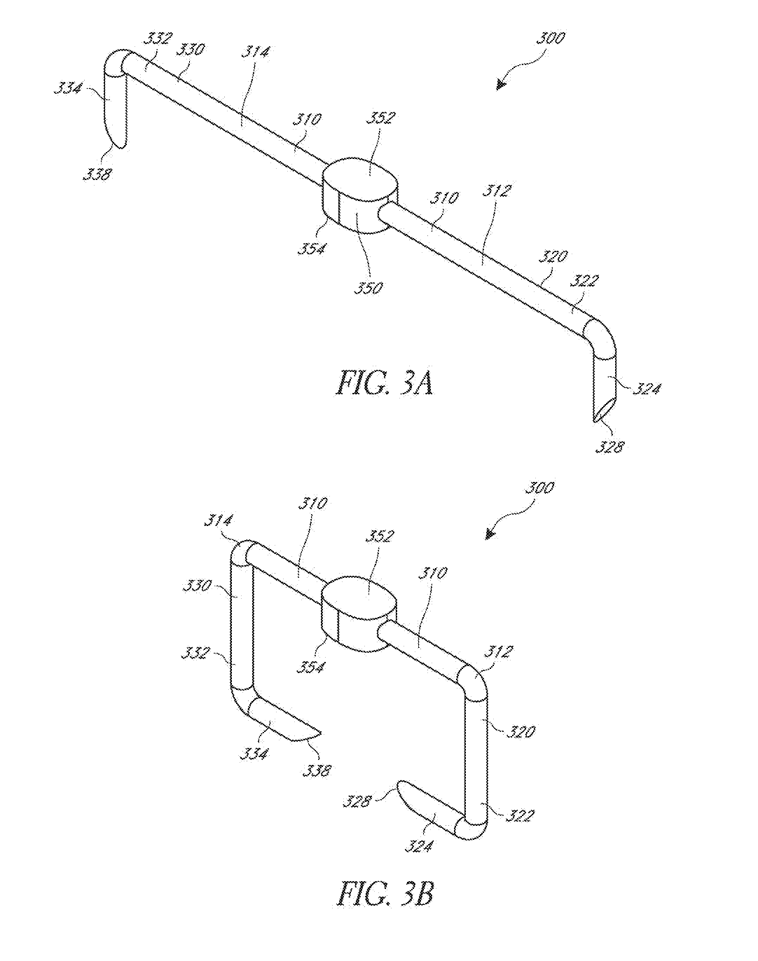

FIGS. 3A and 3B show perspective views of one embodiment of a surgical staple 300, illustrated in a first configuration and a second configuration, respectively. As above, the first configuration may represent an initial configuration and the second configuration may represent a stapled or installed configuration. The staple 300 includes a crown 310 extending between a first end 312 and a second end 314. A first arm 320 extends from the first end 312 of the crown 310. The first arm 320 includes an extension portion 322 and a tooth 324, terminating in a point 328. A second arm 330 extends from the second end 314 of the crown 310. The second arm 330 includes an extension portion 332 and a tooth 334, terminating in a point 338. These features may be substantially similar to similar features discussed above in reference to the staple 100.

The staple 300 also includes a pad 350. The pad 350 may be positioned on the crown 310. The pad 350 may surround a portion of the crown 310. In some embodiments, the pad 350 is positioned at the midpoint of the crown 310 between the first end 312 and the second end 314. In the illustrated embodiment, the pad 350 includes a substantially cylindroid-shaped body. The pad 350 includes an upper surface 352 and a lower surface 354 with the cylindroid-shaped body extending therebetween. The upper surface 352 and/or the lower surface 354 may be substantially flat. The crown 310 may be located in a plane positioned between the upper surface 352 and the lower surface 354. The crown 310 may extend through the cylindroid-shaped body. The crown 310 may extend through a curved surface of the cylindroid-shaped body. Although the pad 350 is illustrated with a cylindroid-shaped body, the pad 350 may include a wide variety of shapes, for example, spheroid shapes, prismatic shapes, etc. Several non-limiting additional shapes for pads will be described in greater detail below.

The pad 350 may be overmolded onto the crown 310. The pad may be made from a material with a hardness which is a material's resistance to permanent indentation, where the hardness may be quantifiable on a shore durometer scale. To measure and quantify hardness of a material, scales of durometer are used where an A scale is for softer plastics, and a D scale is for harder plastics. The A scale and the D scale each may result in a value between 0 and 100. For example, the pad 350 may include one or more soft synthetic polymers of type A shore durometer of 100 or less overmolded onto the crown 310. Some example polymers may include silicone, TPE, or TPU. Alternatively, the pad 350 may include one or more hard synthetic polymers of type D shore durometer of 0 or more overmolded onto the crown 310. Some polymers may include polypropylene, nylon, or ABS. The pad 350 may be integrally formed with the crown 310, for example as a single piece.

The pad 350 may be configured to elevate the crown 310 above the surface of the patient's skin when in use. For example, the bottom surface 354 of the pad 350 may contact the surface of the patient's skin, and the pad 350 may lift the crown 310 above the skin's surface. This may help prevent the staple 300 from becoming ingrown. This may allow the staple 300 to be left in place for longer than a conventional surgical staple, leading to improved wound strength. This may also make the staple 300 easier to remove, for example, because it may be easier to work a removal tool under the crown 310 when the crown is elevated above the skin's surface.

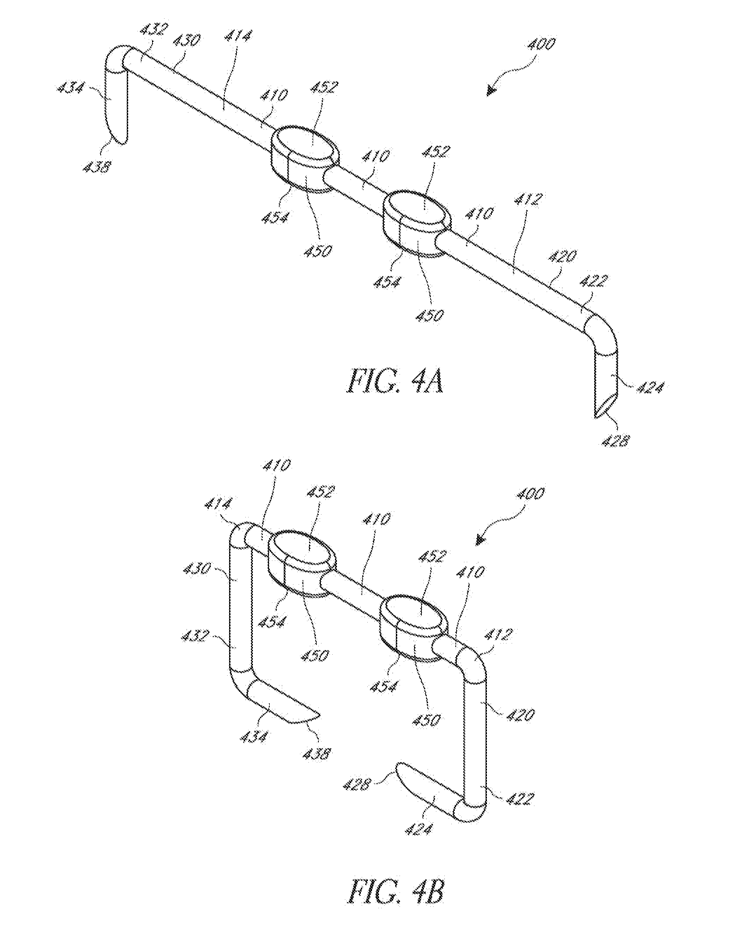

FIGS. 4A and 4B show perspective views of one embodiment of a surgical staple 400, illustrated in a first configuration and a second configuration, respectively. As above, the first configuration may represent an initial configuration and the second configuration may represent a stapled or installed configuration. The staple 400 includes a crown 410 extending between a first end 412 and a second end 414. A first arm 420 extends from the first end 412 of the crown 410. The first arm 420 includes an extension portion 422 and a tooth 424, terminating in a point 428. A second arm 430 extends from the second end 414 of the crown 410. The second arm 430 includes an extension portion 432 and a tooth 434, terminating in a point 438. These features may be substantially similar to similar features discussed above in reference to the staple 100.

The staple 400 also includes two pads 450a, 450b. The pads 450a, 450b may be positioned on the crown 410. The pads 450a, 450b may be spaced apart from each other on the crown 410. Each of the pads 450a, 450b may surround a portion of the crown 410. In the illustrated embodiment, each pad 450a, 450b includes a substantially cylindroid-shaped body. Each pad 450a, 450b includes an upper surface 452 and a lower surface 454 with the cylindroid-shaped body extending therebetween. The upper surfaces 452 and/or the lower surfaces 454 may be substantially flat. Each pad 450a, 450b may include substantially the same width between the upper surface 452 and the lower surface 454. The crown 410 may be located in a plane positioned between the upper surface 452 and the lower surface 454. The crown 410 may extend through the cylindroid-shaped bodies. The crown 410 may extend through a curved surface of the cylindroid-shaped bodies. Although the pads 450a, 450b are each illustrated with a cylindroid-shaped body, the pads 450a, 450b may include a wide variety of shapes, for example, spheroid shapes, prismatic shapes, etc. Several non-limiting additional shapes for pads are described throughout this disclosure. In some embodiments, the pad 450a may include a different shape than the pad 450b.

Each pad 450a, 450b may be overmolded onto the crown 410. For example, the pads 450, 450b may each include silicone overmolded onto the crown 410. The pads 450a, 450b may be integrally formed with the crown 510, for example as a single piece.

The pads 450a, 450b may be configured to elevate the crown 410 above the surface of the patient's skin when in use. For example, the bottom surfaces 454 of the pads 450a, 450b may contact the surface of the patient's skin, and the pads 450a, 450b may lift the crown 410 above the skin's surface. This may help prevent the staple 400 from becoming ingrown. This may allow the staple 400 to be left in place for longer than a conventional surgical staple, leading to improved wound strength. This may also make the staple 400 easier to remove, for example, because it may be easier to work a removal tool under the crown 410 when the crown is elevated above the skin's surface.

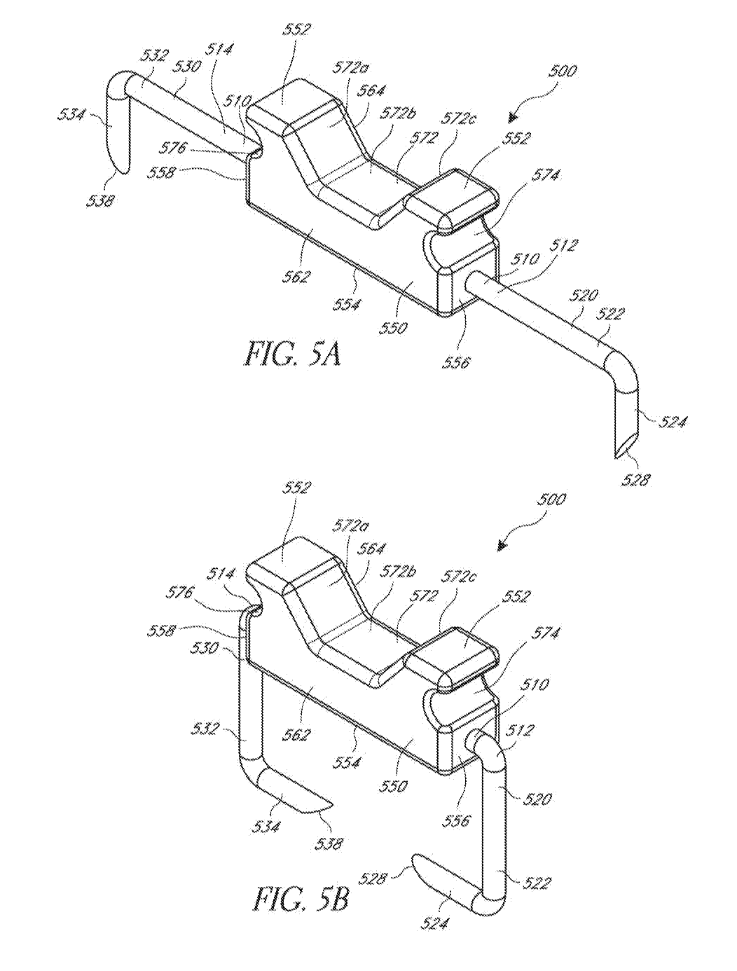

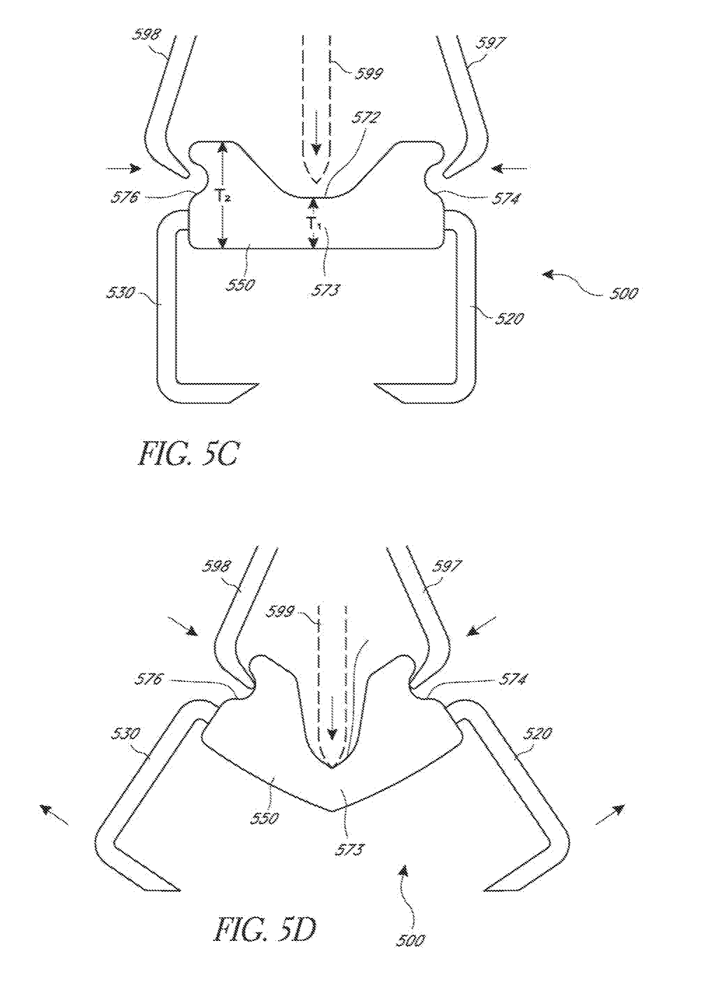

FIGS. 5A and 5B show perspective views of another embodiment of a surgical staple 500, illustrated in a first configuration and a second configuration, respectively. As above, the first configuration may represent an initial configuration and the second configuration may represent a stapled or installed configuration. The staple 500 includes a crown 510 extending between a first end 512 and a second end 514. A first arm 520 extends from the first end 512 of the crown 510. The first arm 520 includes an extension portion 522 and a tooth 524, terminating in a point 528. A second arm 530 extends from the second end 514 of the crown 510. The second arm 530 includes an extension portion 532 and a tooth 534, terminating in a point 538. These features may be substantially similar to similar features discussed above in reference to the staple 100.

The staple 500 also includes a pad 550. The pad 550 may be positioned on the crown 510. The pad 550 may surround a portion of the crown 510. The pad 550 may be centered on the crown between the first end 512 and the second end 514. In the illustrated embodiment, the pad 550 extends substantially the entire length of the crown 510, although the pad 550 may be shorter in some embodiments. In the illustrated embodiment, the pad 550 includes a substantially prismatic-shaped body, having a top surface 552, a bottom surface 554, a first side surface 556, a second side surface 558, a front surface 562, and a back surface 564. Each surface may be orthogonal to each adjacent surface, although this need not be the case in all embodiments. In some embodiments, the surfaces are substantially flat. In some embodiments, one or more of the surfaces may be curved. The pad 550 may be oriented such that the bottom surface 554 contacts a patient's skin when in use.

A notch 572 may be formed into the body of the pad 550 from the top surface 552. The notch may be formed as a first angled surface 572a, angling from the top surface 552 to central surface 572b. An opposite side of the central surface 572b may be connected to second angled surface 572c, which angles back to the top surface 552. The angles of the angled surfaces 572a, 572c may be approximately 90 degrees or less than approximately 90 degrees. In some embodiments, the angles are between approximately 30 degrees and approximately 60 degrees. In some embodiments the angles, are approximately 45 degrees.

The notch 572 may extend entirely through the body from the front surface 562 to the back surface along an axis that may be substantially orthogonal to the crown 510. The notch may include a V-shape with a substantially flat bottom. As will be described below in reference to FIGS. 5C and 5D, the notch 572 creates a thinner section of the pad 550. The thinner section may be configured to bend to allow removal of the staple 500.

A first groove 574 is formed into the body of the pad 550 from the first side 556. The first groove 574 may extend entirely through the body from the front surface 562 to the back surface 564 along an axis substantially orthogonal to the crown. The first groove 574 may include a rounded groove. The first groove 574 may be positioned on the first side 556 above a point where the crown 510 exits the pad 550. A second groove 576 is formed into the body of the pad 550 from the first side 558. The second groove 576 may extend entirely through the body from the front surface 562 to the back surface 564 along an axis substantially orthogonal to the crown. The second groove 576 may include a rounded groove. The second groove 576 may be positioned on the second side 558 above a point where the crown 510 exits the pad 550. In some embodiments, the first groove 574 and the second groove 576 extend along parallel axes. As will be shown and described in reference to FIGS. 5C and 5D below, the grooves 574, 576 may be configured to receive a portion of a removal tool during removal of the staple 500.

The pad 550 may be overmolded onto the crown 510. For example, the pad 550 may include silicone overmolded onto the crown 510. The pad 550 may be integrally formed with the crown 510, for example as a single piece. In some embodiments, the pad 550 is made from a stiff resin, such as, for example, polypropylene, nylon, or ABS. In some embodiments, the crown 510 may not extend entirely through the pad 550. That is, the crown 550 may be divided into two parts: a first part connected to the first arm 520 and a second part connected to the second arm 530. The first part and the second part of the crown 510 may be spaced apart within the pad 550.

The pad 550 may be configured to elevate the crown 510 above the surface of the patient's skin when in use. For example, the bottom surface 554 of the pad 550 may contact the surface of the patient's skin, and the pad 550 may lift the crown 510 above the skin's surface. This may help prevent the staple 500 from becoming ingrown. This may allow the staple 500 to be left in place for longer than a conventional surgical staple, leading to improved wound strength.

FIGS. 5C and 5D show front views illustrating how the surgical staple 500 may be removed in some embodiments. As discussed above, the staple 500 includes a pad 550, which can be made from a soft or hard resin overmolded on the crown. The pad 550 includes a notch 572 formed in the top surface thereof and a groove 574, 576 formed on opposing side surfaces. These features may facilitate removal of the staple 500. As shown in FIG. 5C, the notch 572 narrows the thickness of the pad 550 such that a central portion 573 has a smaller thickness T₁ than the edge portions, which have a thickness T₂. The central portion 573 may thus be configured to bend to allow the staple 500 to be removed. In some embodiments, the central portion 573 acts as a living hinge.

FIG. 5C shows the configuration of the staple 500 in use (for example, closing a patient's wound). To remove the staple 500, opposing legs 597, 598 of a removal tool may be engaged with grooves 574, 576 respectively. In some embodiments, the removal tool may be a hemostat, forceps, pliers, etc. Although not necessary in all embodiments, a central leg 599 (which may comprise part of a separate tool or the same removal tool) is brought into contact with the notch 572. As shown in FIG. 5D, the opposing legs 597, 598 can be brought together (towards the center of the staple 500). This causes the staple 500 to bend at the central portion 573. As the staple 500 bends, the arms 520, 530 rotate outward disengaging from the patient's skin. In some embodiments, the central leg 599 can be pressed downward into the notch 572 at the same time, although this is not required. This procedure advantageously allows the staple 500 to be removed without requiring a removal tool to be worked between the staple 500 and the patient's skin.

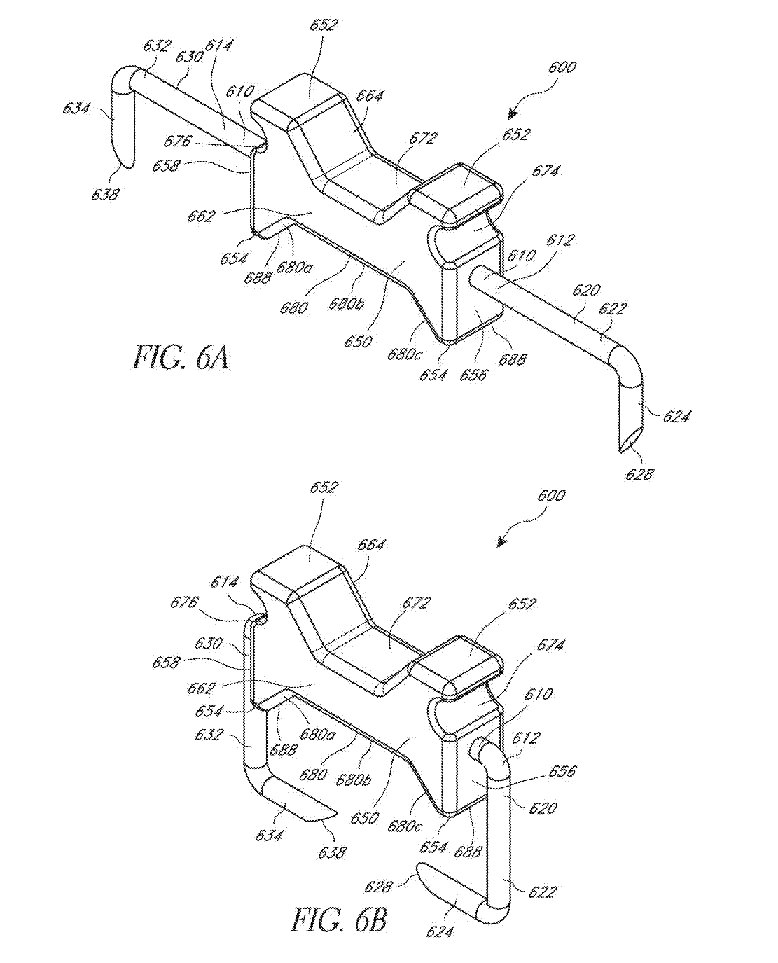

FIGS. 6A and 6B show perspective views of another embodiment of a surgical staple 600, illustrated in a first configuration and a second configuration, respectively. As above, the first configuration may represent an initial configuration and the second configuration may represent a stapled or installed configuration. The staple 600 includes a crown 610 extending between a first end 612 and a second end 614. A first arm 620 extends from the first end 612 of the crown 610. The first arm 620 includes an extension portion 622 and a tooth 624, terminating in a point 628. A second arm 630 extends from the second end 614 of the crown 610. The second arm 630 includes an extension portion 632 and a tooth 634, terminating in a point 638. These features may be substantially similar to similar features discussed above in reference to the staple 100.

The staple 600 also includes a pad 650. Similar to the pad 550 described above, the pad 660 may be positioned on the crown 610 and may include a substantially prismatic-shaped body, having a top surface 652, a bottom surface 654, a first side surface 656, a second side surface 658, a front surface 662, and a back surface 664. The pad 650 may also include a notch 672 and grooves 674, 676, which all may be substantially similar to similar features of the pad 550, described above. The notch 672 and grooves 674, 676 may allow the staple 600 to be removed as described above in reference to FIGS. 5A and 5B.

The pad 650 also includes an eversion recess 680 formed into the body of the pad 650 from the bottom surface 654 thereof. The eversion recess 680 may be formed as a channel extending through the body of the pad 650 from the front surface 662 to the back surface 664. The eversion recess 680 may include a first angled surface 680a, angling from the bottom surface 654 to central surface 680b. An opposite side of the central surface 580b may be connected to second angled surface 680c, which angles back to the bottom surface 554. The angles of the angled surfaces 680a, 608c may be approximately 90 degrees or less than approximately 90 degrees. In some embodiments, the angles are between approximately 30 degrees and approximately 60 degrees. In some embodiments the angles, are approximately 45 degrees. The eversion recess 680 forms an indentation in the bottom surface 654, forming feet 688 on each side thereof. The eversion recess 680 creates a space for wound eversion as described above. In some embodiments, the eversion recess 680 contacts the incision. In some embodiments, the eversion recess 680 does not contact the incision. The feet 688 may contact the skin's surface on either side of the region of wound eversion.

The pad 650 may be overmolded onto the crown 610. For example, the pad 650 may include silicone overmolded onto the crown 610. The pad 650 may be integrally formed with the crown 610, for example as a single piece. In some embodiments, the pad 650 is made from a stiff resin, such as, for example, polypropylene. In some embodiments, the crown 610 may not extend entirely through the pad 650. That is, the crown 650 may be divided into two parts: a first part connected to the first arm 620 and a second part connected to the second arm 630. The first part and the second part of the crown may be spaced apart within the pad 650.

The pad 650 may be configured to elevate the crown 610 above the surface of the patient's skin when in use. For example, the bottom surface 654 of the feet 688 of the pad 650 may contact the surface of the patient's skin, and the pad 650 may lift the crown 610 above the skin's surface. This may help prevent the staple 600 from becoming ingrown. This may allow the staple 600 to be left in place for longer than a conventional surgical staple, leading to improved wound strength. The eversion recess 680 may create a space for wound eversion.

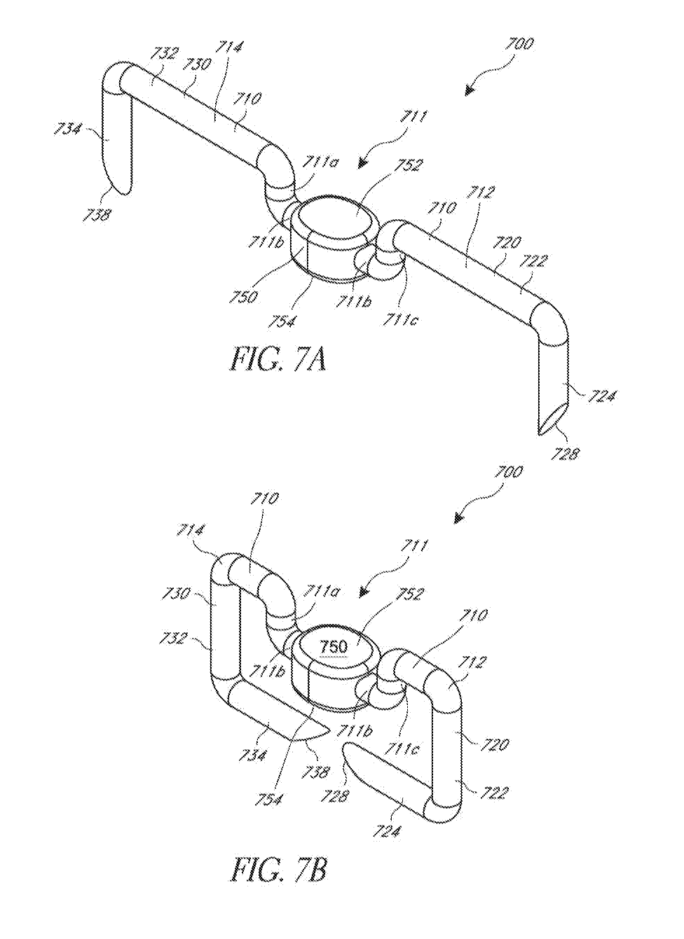

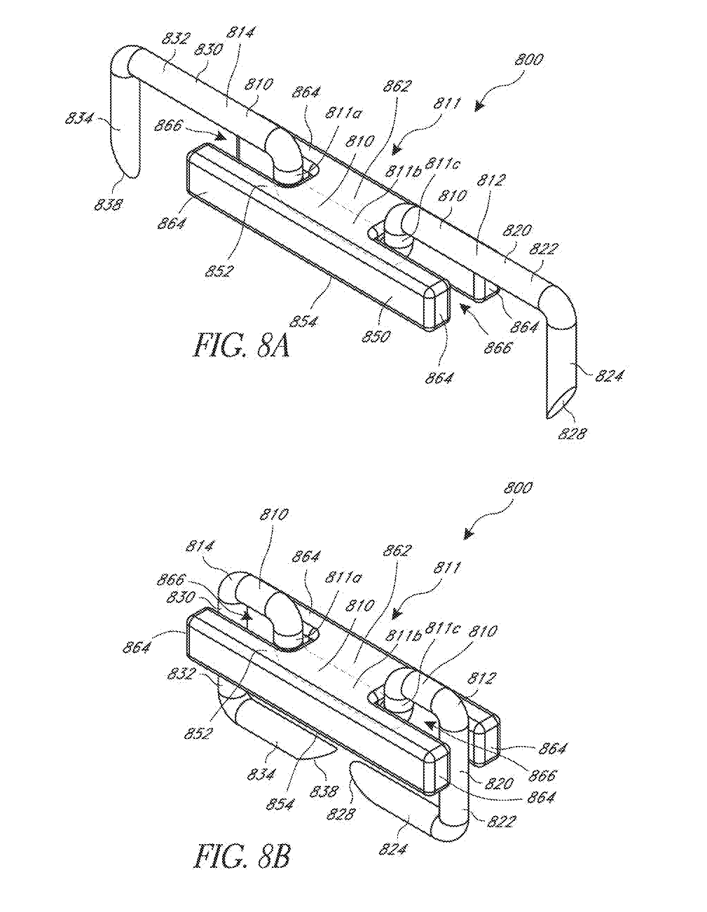

FIGS. 7A and 7B show perspective views of another embodiment of a surgical staple 700, illustrated in a first configuration and a second configuration, respectively. As above, the first configuration may represent an initial configuration and the second configuration may represent a stapled or installed configuration. The staple 700 includes a crown 710 extending between a first end 712 and a second end 714. However, unlike the embodiments illustrated in FIGS. 3A-6B, the crown 710 does not extend linearly in the embodiment of the staple 700. As shown, the crown 710 includes a U-shaped portion 711. The U-shaped portion 711 may be located substantially at the midpoint between the first end 712 and the second end 711. The U-shaped portion may include legs 711a, 711c that extend at an angle relative an axis extending between the first end 712 and the second end 714. The angle may be approximately 90 degrees. In some embodiments, the angle may be less than 90 degrees. A connecting portion 711b extends between the legs 711a, 711c. The connecting portion 711b may be oriented so as to be substantially parallel with the axis extending between the first end 712 and the second end 714. In some embodiments, the connecting portion 711b and/or the legs 711a, 711c may include curved portions. The U-shaped portion 711 may extend generally in the same direction as the extension portions 722, 732 of the legs 720, 730 when the staple 700 is in the second configuration.

A first arm 720 extends from the first end 712 of the crown 710. The first arm 720 includes an extension portion 722 and a tooth 724 terminating in a point 728. A second arm 730 extends from the second end 714 of the crown 710. The second arm 730 includes an extension portion 732 and a tooth 734 terminating in a point 738. These features may be substantially similar to similar features discussed above in reference to the staple 100.

The staple 700 also includes a pad 750. The pad 750 may be positioned on the crown 710. The pad 750 may be positioned on the U-shaped portion. In the illustrated embodiment, the pad 750 is positioned on the connecting portion 711b of the U-shaped portion 711. The pad 750 may surround a portion of the crown 710. In the illustrated embodiment, the pad 750 includes a substantially cylindroid-shaped body. The pad 750 includes an upper surface 752 and a lower surface 754 with the cylindroid-shaped body extending therebetween. The upper surface 752 and/or the lower surface 754 may be substantially flat. The connecting portion 711b may be located in a plane positioned between the upper surface 752 and the lower surface 754. The crown 710 may extend through the cylindroid-shaped body. The crown 710 may extend through a curved surface of the cylindroid-shaped body. Although the pad 750 is illustrated with a cylindroid-shaped body, the pad 750 may include a wide variety of shapes, for example, spheroid shapes, prismatic shapes, etc. Several non-limiting additional shapes for pads will be described throughout this application. The pad 750 may be overmolded onto the crown 710. For example, the pad 750 may include silicone overmolded onto the crown 710. The pad 750 may be integrally formed with the crown 710, for example as a single piece. In some embodiments, the pad 750 may be omitted.

The U-shaped portion 711 of the crown 710 may be configured to elevate at least a portion of the crown 710 above the surface of the patient's skin when in use. For example, the connecting portion 711b may contact the surface of the patient's skin, and the legs 711a, 711c may lift the remainder of the crown 710 above the skin's surface. If a pad 750 is included, the pad 750 may further this effect, lifting the crown 710 above the surface of the skin. This may help prevent the staple 700 from becoming ingrown. This may allow the staple 700 to be left in place for longer than a conventional surgical staple, leading to improved wound strength. This may also make the staple 700 easier to remove, for example, because it may be easier to work a removal tool under the crown 710 when the crown is elevated above the skin's surface.

FIGS. 8A and 8B show perspective views of another embodiment of a surgical staple 800, illustrated in a first configuration and a second configuration, respectively. As above, the first configuration may represent an initial configuration and the second configuration may represent a stapled or installed configuration. The staple 800 includes a crown 810 shaped substantially similarly to the crown 710 described above. That is, the crown 810 includes a U-shaped portion 811. The U-shaped portion 811 may be located substantially at the midpoint between the first end 812 and the second end 811. The U-shaped portion may include legs 811a, 811c that extend at an angle relative an axis extending between the first end 812 and the second end 814. The angle may be approximately 90 degrees. In some embodiments, the angle may be less than 90 degrees. A connecting portion 811b extends between the legs 811a, 811c. The connecting portion 811b may be oriented so as to be substantially parallel with the axis extending between the first end 812 and the second end 814. In some embodiments, the connecting portion 811b and/or the legs 811a, 811c may include curved portions. The U-shaped portion 811 may extend generally in the same direction as the extension portions 822, 832 of the legs 820, 830 when the staple 800 is in the second configuration.

The staple 800 also includes a first arm 820 extends from the first end 812 of the crown 810. The first arm 820 includes an extension portion 822 and a tooth 824 terminating in a point 828. A second arm 830 extends from the second end 814 of the crown 810. The second arm 830 includes an extension portion 832 and a tooth 834 terminating in a point 838. These features may be substantially similar to similar features discussed above in reference to the staple 100.

The staple 800 also includes a pad 850. The pad 850 may be positioned on the crown 810. The pad 850 may be positioned on the U-shaped portion. In the illustrated embodiment, the pad 850 is positioned on the connecting portion 811b of the U-shaped portion 811. The pad 850 may surround a portion of the crown 810. The pad 850 includes an H-shape, including a central portion 862 with four extension portions 864 extending therefrom. Two of the extension portions 864 extend in a first direction. A slot 866 is formed between the two extension portions 864. The other two of the extensions portions 864 extend in a second direction, substantially opposite the first direction. A slot 866 is formed between these two extension portions 864. Therefore the pad 850 has an H-shape. The crown 810 may extend through the central portion 862 of the H-shape. The legs 811a, 811c may extend through the slots 866. When the staple 800 is in the second configuration, the legs 820, 830 may also extend through the slots 866. The pad 850 may also include an upper surface 852 and a lower surface 854. The pad 850 may be overmolded onto the crown 810. For example, the pad 850 may include silicone overmolded onto the crown 810. The pad 850 may be integrally formed with the crown 810, for example as a single piece.

As with the staple 700 described above, The U-shaped portion 811 of the crown 810 may be configured to elevate at least a portion of the crown 810 above the surface of the patient's skin when in use. For example, the connecting portion 811b may contact the surface of the patient's skin, and the legs 811a, 811c may lift the remainder of the crown 810 above the skin's surface. The pad 850 may further this effect, lifting the crown 810 above the surface of the skin. The H-shape of the pad 850 provides a large contact surface between the bottom surface 854 of the pad and the skin. This may help prevent the staple 800 from becoming ingrown. This may allow the staple 800 to be left in place for longer than a conventional surgical staple, leading to improved wound strength. This may also make the staple 800 easier to remove, for example, because it may be easier to work a removal tool under the crown 810 when the crown is elevated above the skin's surface.

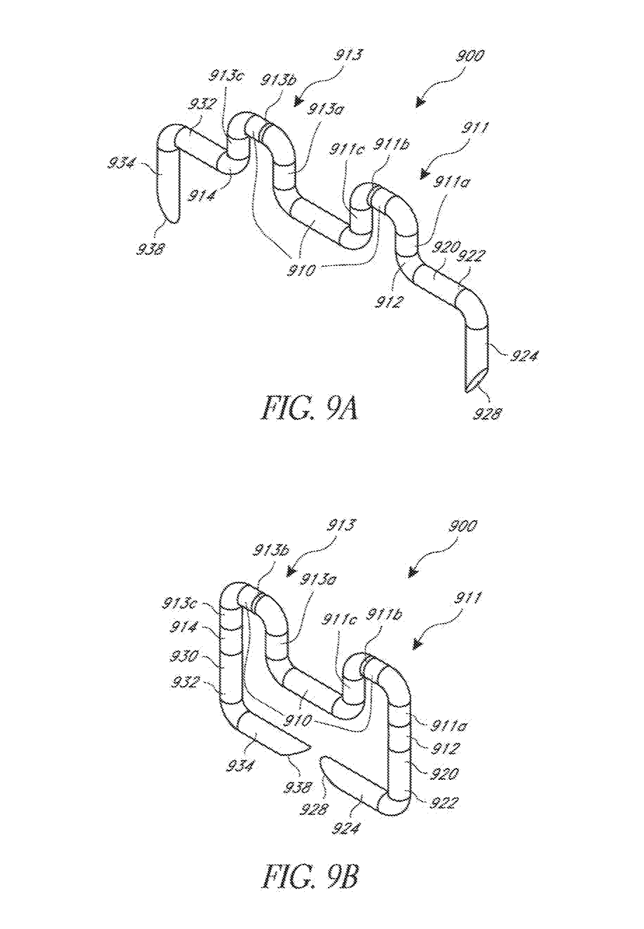

FIGS. 9A and 9B show perspective views of another embodiment of a surgical staple 900, illustrated in a first configuration and a second configuration, respectively. As above, the first configuration may represent an initial configuration and the second configuration may represent a stapled or installed configuration. The staple 900 includes a crown 910 extending between a first end 912 and a second end 914. However, the crown 910 does not extend linearly in the embodiment of the staple 900. As shown, the crown 910 includes a first U-shaped portion 911 and a second U-shaped portion 913. The first U-shaped portion 911 may be positioned on the crown 910 adjacent to the first end 912. The second U-shaped portion 913 may be positioned on the crown 910 adjacent to the second end 914.

The first U-shaped portion 911 may include legs 911a, 911c that extend at an angle relative an axis extending between the first end 912 and the second end 914. The angle may be approximately 90 degrees. In some embodiments, the angle may be less than 90 degrees. A connecting portion 911b extends between the legs 911a, 911c. The connecting portion 911b may be oriented so as to be substantially parallel with the axis extending between the first end 912 and the second end 914. In some embodiments, the connecting portion 911b and/or the legs 911a, 911c may include curved portions. The second U-shaped portion 913 may include legs 913a, 913c that extend at an angle relative an axis extending between the first end 912 and the second end 914. The angle may be approximately 90 degrees. In some embodiments, the angle may be less than 90 degrees. A connecting portion 913b extends between the legs 913a, 913c. The connecting portion 913b may be oriented so as to be substantially parallel with the axis extending between the first end 912 and the second end 914. In some embodiments, the connecting portion 913b and/or the legs 913a, 913c may include curved portions.

The first and second U-shaped portions 911, 913 may extend generally in the same direction. For example, in the illustrated embodiment, the first and second U-shaped portions 911, 913 extend generally in a direction that is opposite the direction of the extension portions 922, 932 of the legs 920, 930 when the staple 900 is in the second configuration. A central portion 910a of the crown 910 extends between the U-shaped portions. The central portion 910a may lie on the axis extending between the first end 912 and the second end 914.

The staple 900 also includes a first arm 920 extends from the first end 912 of the crown 910. The first arm 920 includes an extension portion 922 and a tooth 924 terminating in a point 928. A second arm 930 extends from the second end 914 of the crown 910. The second arm 930 includes an extension portion 932 and a tooth 934 terminating in a point 938. These features may be substantially similar to similar features discussed above in reference to the staple 100.

The U-shaped portions 911, 913 of the crown 910 may be configured to elevate at least a portion of the crown 910 above the surface of the patient's skin when in use. For example, the central portion 910a of the crown 910 may contact the surface of the patient's skin, and the U-shaped portions 911, 913 may lift the remainder of the crown 910 above the skin's surface. This may help prevent the staple 900 from becoming ingrown. This may allow the staple 900 to be left in place for longer than a conventional surgical staple, leading to improved wound strength. This may also make the staple 900 easier to remove, for example, because it may be easier to work a removal tool under the crown 910 when the crown is elevated above the skin's surface.

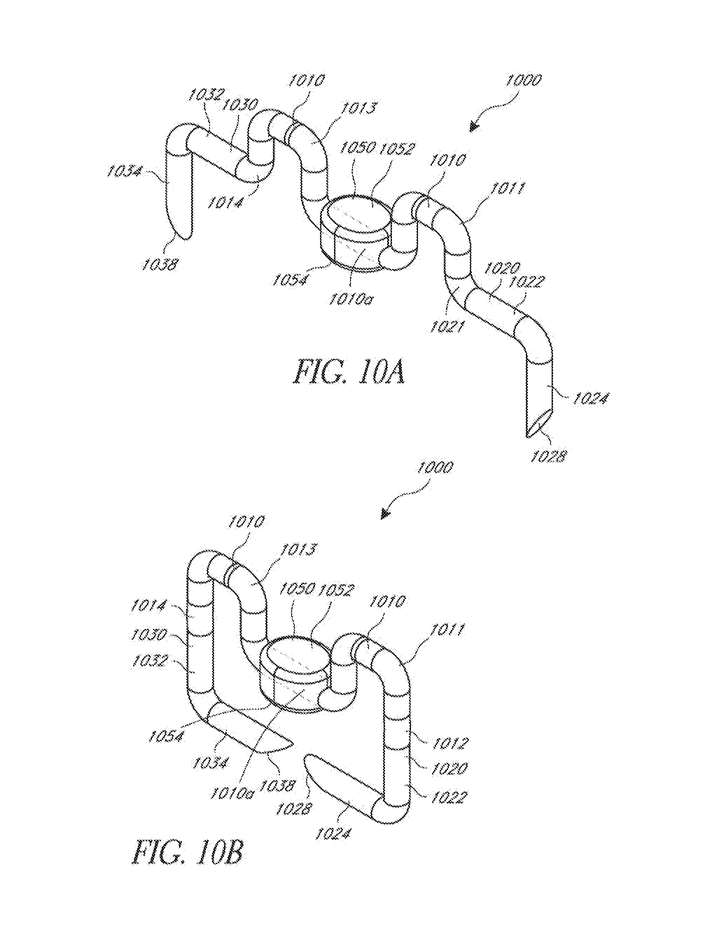

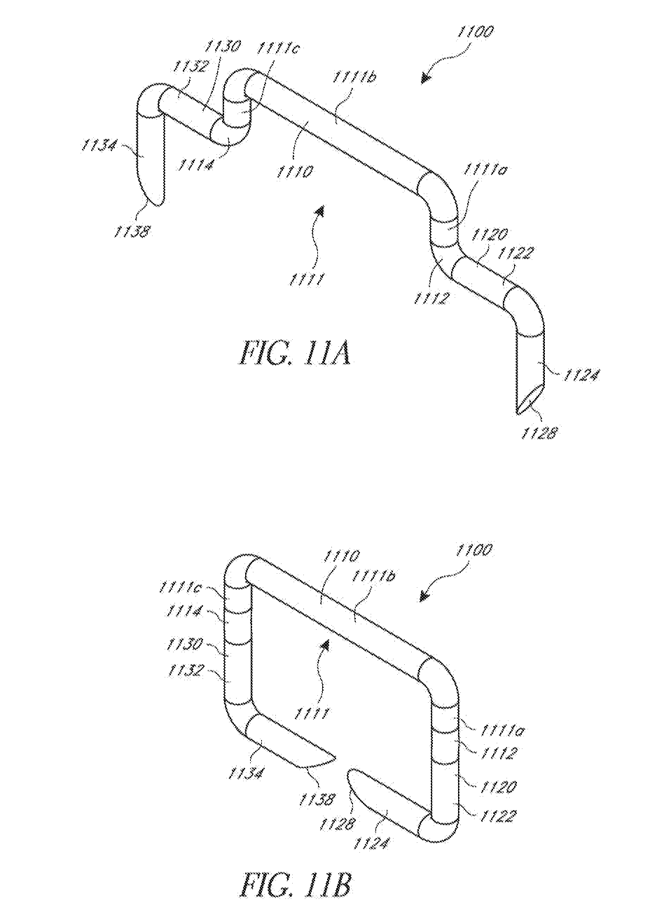

FIGS. 10A and 10B show perspective views of an embodiment of a surgical staple 1000, illustrated in a first configuration and a second configuration, respectively. The surgical staple 1000 is substantially similar to the surgical staple 900 described above, except the surgical staple 1000 includes a pad. That is, the surgical staple 1000 includes a crown 1010 with first and second U-shaped portions 1011, 1013 separated by a central portion 1010a of the crown 1010 substantially similar to those described above. The staple 1000 also includes a pad 1050. The pad 1050 may be positioned on the crown 1010. The pad 1050 may be positioned on the central portion 1010a. The pad 1050 may surround a portion of the crown 1010. In the illustrated embodiment, the pad 1050 includes a substantially cylindroid-shaped body. The pad 1050 includes an upper surface 1052 and a lower surface 1054 with the cylindroid-shaped body extending therebetween. The upper surface 1052 and/or the lower surface 1054 may be substantially flat. The central portion 1010a may be located in a plane positioned between the upper surface 1052 and the lower surface 1054. The crown 1010 may extend through the cylindroid-shaped body. The crown 1010 may extend through a curved surface of the cylindroid-shaped body. Although the pad 1050 is illustrated with a cylindroid-shaped body, the pad 1050 may include a wide variety of shapes, for example, spheroid shapes, prismatic shapes, etc. Several non-limiting additional shapes for pads are described throughout this application. The pad 1050 may be overmolded onto the crown 1010. For example, the pad 1050 may include silicone overmolded onto the crown 1010. The pad 1050 may be integrally formed with the crown 710, for example as a single piece.

In conjunction with the U-shaped portions 1011, 1013, the pad 1050 may be configured to elevate at least a portion of the crown 1010 above the surface of the patient's skin when in use. For example, the bottom surface 1054 of the pad 1050 may contact the surface of the patient's skin, and the U-shaped portions 1011, 1013 may lift the remainder of the crown 1010 above the skin's surface. This may help prevent the staple 1000 from becoming ingrown. This may allow the staple 1000 to be left in place for longer than a conventional surgical staple, leading to improved wound strength. This may also make the staple 1000 easier to remove, for example, because it may be easier to work a removal tool under the crown 1010 when the crown is elevated above the skin's surface.

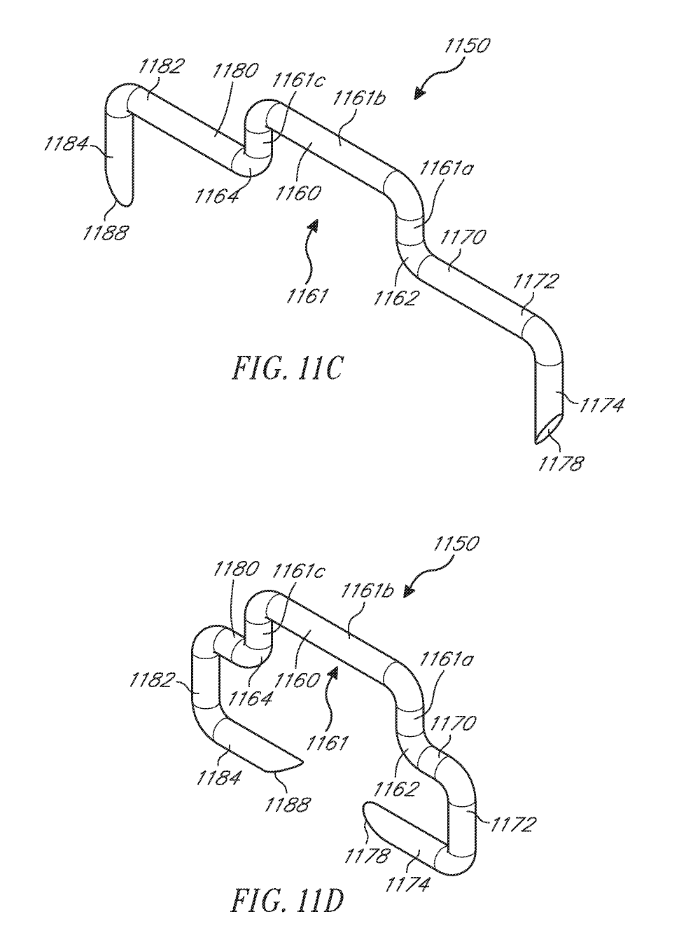

FIGS. 11A and 11B show perspective views of another embodiment of a surgical staple 1100, illustrated in a first configuration and a second configuration, respectively. As above, the first configuration may represent an initial configuration and the second configuration may represent a stapled or installed configuration. The staple 1100 includes a crown 1110 extending between a first end 1112 and a second end 1114. The crown 1110 includes a U-shape 1111. The U-shape 1111 may include legs 1111a, 1111c that extend at an angle relative an axis extending between the first end 1112 and the second end 1114. The angle may be approximately 90 degrees. In some embodiments, the angle may be less than 90 degrees. A connecting portion 1111b extends between the legs 1111a, 1111c. The connecting portion 1111b may be oriented so as to be substantially parallel with the axis extending between the first end 1112 and the second end 1114. In some embodiments, the connecting portion 1111b and/or the legs 1111a, 1111c may include curved portions. The U-shape 1111 may extend generally in a direction opposite the direction of the extension portions 1122, 1132 of the legs 1120, 1130 when the staple 1100 is in the second configuration. The staple 1100 also includes a first arm 1120 that extends from the first end 1112 of the crown 1110. The first arm 1120 includes an extension portion 1122 and a tooth 1124 terminating in a point 1128. A second arm 1130 extends from the second end 1114 of the crown 1110. The second arm 1130 includes an extension portion 1132 and a tooth 1134 terminating in a point 1138. These features may be substantially similar to similar features discussed above in reference to the staple 100.

FIGS. 11C and 11D show perspective views of another embodiment of a surgical staple 1150, illustrated in a first configuration and a second configuration, respectively. As above, the first configuration may represent an initial configuration and the second configuration may represent a stapled or installed configuration. The staple 1150 includes a crown 1160 extending between a first end 1162 and a second end 1164. The crown 1160 includes a U-shape 1161. The U-shape 1161 may include legs 1161a, 1161c that extend at an angle relative an axis extending between the first end 1162 and the second end 1164. The angle may be approximately 90 degrees. In some embodiments, the angle may be less than 90 degrees. A connecting portion 1161b extends between the legs 1161a, 1161c. The connecting portion 1161b may be oriented so as to be substantially parallel with the axis extending between the first end 1162 and the second end 1164. In some embodiments, the connecting portion 1161b and/or the legs 1161a, 1161c may include curved portions. The U-shape 1161 may extend generally in a direction opposite the direction of the extension portions 1172, 1182 of the legs 1170, 1180 when the staple 1150 is in the second configuration. The staple 1150 also includes a first arm 1170 that extends from the first end 1162 of the crown 1160. The first arm 1170 includes an extension portion 1172 and a tooth 1174 terminating in a point 1178. A second arm 1180 extends from the second end 1164 of the crown 1160. The second arm 1180 includes an extension portion 1182 and a tooth 1184 terminating in a point 1188. These features may be substantially similar to similar features discussed above in reference to the staple 100.

The U-shape 1161 of the crown 1160 may be configured to elevate at least a portion of the crown 1160 above the surface of the patient's skin when in use. For example, the connecting portion 1161b may be elevated above the skin's surface. This may help prevent the staple 1150 from becoming ingrown. This may allow the staple 1150 to be left in place for longer than a conventional surgical staple, leading to improved wound strength. This may also make the staple 1150 easier to remove, for example, because it may be easier to work a removal tool under the crown 1160 when the crown is elevated above the skin's surface.

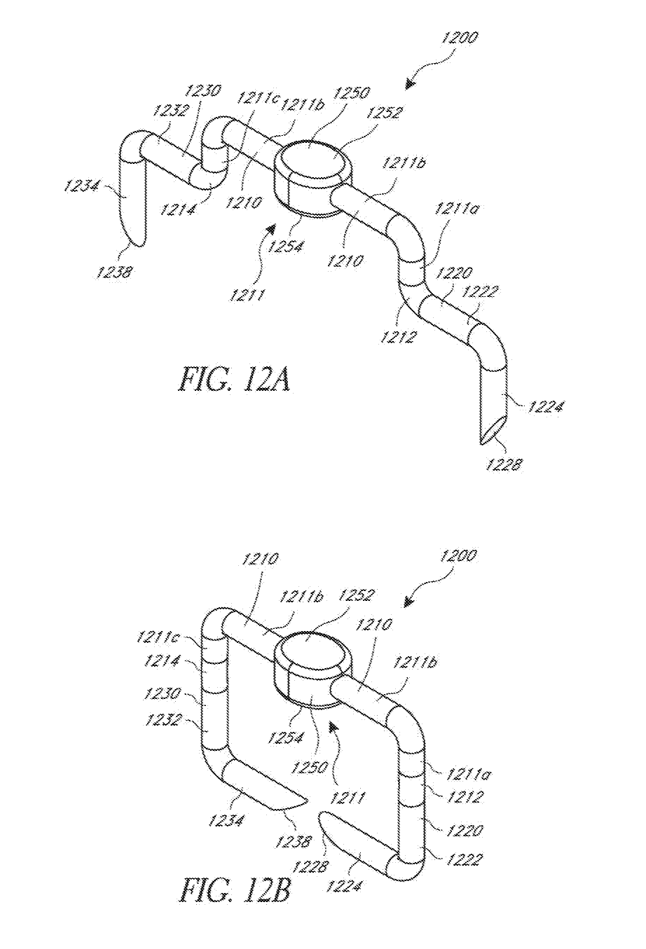

FIGS. 12A and 12B show perspective views of an embodiment of a surgical staple 1200, illustrated in a first configuration and a second configuration, respectively. The surgical staple 1200 is substantially similar to the surgical staple 1100 described above, except the surgical staple 1200 includes a pad 1250. That is, the surgical staple 1200 includes a crown 1210 with a U-shape 1211, including legs 1211a, 1211c connected by a connecting portion 1211b similar to those described above. The staple 1200 also includes a pad 1250. The pad 1250 may be positioned on the crown 1210. The pad 1250 may be positioned on the connecting portion 1211b. The pad 1250 may surround a portion of the crown 1210. In the illustrated embodiment, the pad 1250 includes a substantially cylindroid-shaped body. The pad 1250 includes an upper surface 1252 and a lower surface 1254 with the cylindroid-shaped body extending therebetween. The upper surface 1252 and/or the lower surface 1254 may be substantially flat. The connecting portion 1210b may be located in a plane positioned between the upper surface 1252 and the lower surface 1254. The crown 1210 may extend through the cylindroid-shaped body. The crown 1210 may extend through a curved surface of the cylindroid-shaped body. Although the pad 1250 is illustrated with a cylindroid-shaped body, the pad 1250 may include a wide variety of shapes, for example, spheroid shapes, prismatic shapes, etc. Several non-limiting additional shapes for pads are described throughout this application. The pad 1250 may be overmolded onto the crown 1210. For example, the pad 1250 may include or be formed of silicone overmolded onto the crown 1210. The pad 1250 may be integrally formed with the crown 710, for example as a single piece.

In conjunction with the U-shaped 1211 of the crown 1210, the pad 1250 may be configured to elevate at least a portion of the crown 1210 above the surface of the patient's skin when in use. For example, the bottom surface 1254 of the pad 1250 may contact the surface of the patient's skin, and the U-shape 1211 may lift the remainder of the crown 1210 above the skin's surface. This may help prevent the staple 1200 from becoming ingrown. This may allow the staple 1200 to be left in place for longer than a conventional surgical staple, leading to improved wound strength. This may also make the staple 1200 easier to remove, for example, because it may be easier to work a removal tool under the crown 1210 when the crown is elevated above the skin's surface.

Although several embodiments of surgical staples have been illustrated in the figures and described in the text, these embodiments are provided by way of example only. The features of these embodiments may be altered, modified, combined, etc. in a variety of ways which will be apparent to one of skill in the art upon review and implementation of this disclosure.

The remaining figures show views of various embodiments of pressure relief devices. As will become apparent from the following description, the pressure relief devices can be used with staples and/or various types of sutures to close an incision. The pressure relief devices are configured to elevate the external portions of the staples and/or sutures above the surface of the skin. This may reduce pressure on the incision and/or reduce the likelihood that the staples and/or sutures will become ingrown. In some instances, use of a pressure relief device may allow the staples and/or sutures to remain in place for longer than when the staples and/or sutures are used alone and the external portion thereof rest directly on the patient's skin. Sutures used with the pressure relief devices may be secured with an external device, such as any of the suture securing devices described in U.S. patent application Ser. No. 14/990,715, filed on Jan. 7, 2016 (the disclosure of which incorporated herein by reference in its entirety), or through conventional methods, such as the tying of a surgical knot. Pressure relief devices may include a body with a shape configured to match the shape of a staple, for example a width of a staple, and/or a stitching pattern for one or more sutures, for example, simple interrupted, horizontal mattress, or vertical mattress sutures. The shape may include one or more slots and/or openings formed through the body. The body may be positioned across or proximal to a wound or incision. In use, external portions of the staple and/or suture stitch pass over the body of the pressure relief device. Thus, the body of the pressure relief device is positioned between the surface of the patient's skin and the external portions of the sutures. In some embodiments, the staples and/or sutures themselves may secure the pressure relief device in place, in some embodiments.

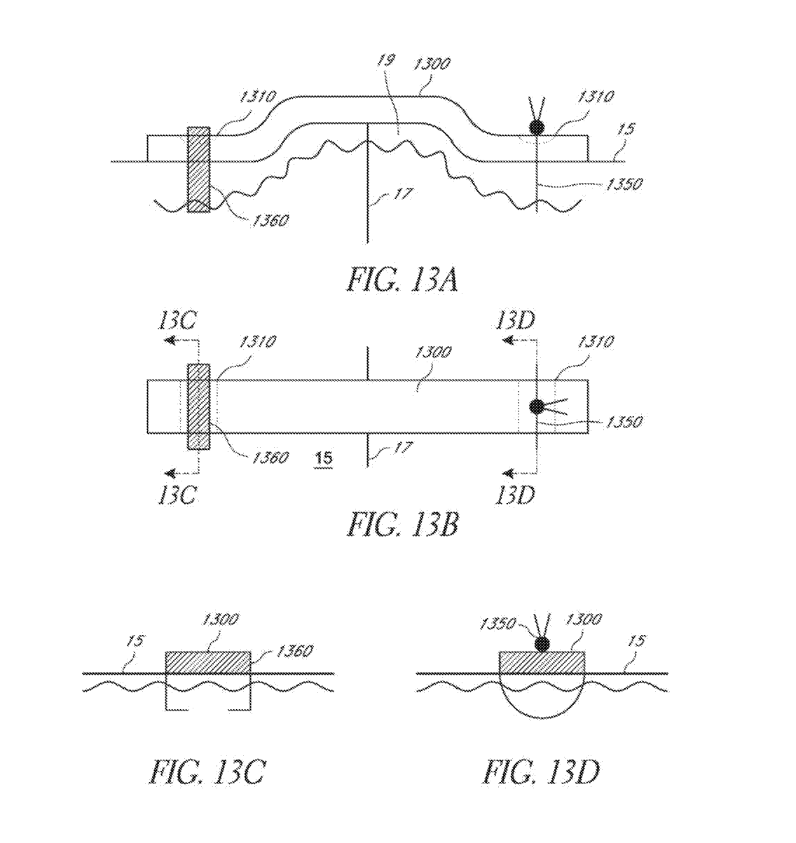

FIGS. 13A and 13B show side and top views of one embodiment of a pressure relief device 1300. The pressure relief device 1300 includes a substantially rectangular body. In the illustrated embodiment, the pressure relief device 1300 is positioned length-wise across an incision 17 on the surface of a patient's skin 15. In other embodiments, the pressure relief device 1300 may be positioned width-wise across the incision. The body 1300 may be made from a flexible material, for example, a medical grade rubber or silicone. The body 1300 may thus conform to the shape of the skin 15, including, for example, conforming to the shape of a region of wound eversion 19. In some embodiments, the body 1300 may be made from a rigid material.

The pressure relief device 1300 includes features formed therein to receive external portions of a suture 1350 and/or a staple 1360. In the illustrated embodiment, the features are grooves 1310 extending across the width of the pressure relief device 1300. The thickness of the grooves 1310 may correspond to the thickness of a suture thread or a surgical staple. As shown, the staple 1360 is installed over the pressure relief device 1300, with the external portion of the staple 1360 resting in the groove 1310. See, for example, the cross-sectional view of FIG. 13C. Similarly, the suture 1350 is formed over the pressure relief device 1300, with the external portion of the suture resting in the groove 1310. See, for example, the cross-sectional view of FIG. 13D.

The pressure relief device 1300 may be used in a variety of ways to close an incision. For example, an example method for closing an incision may include positioning the pressure relief device 1300 proximal to a wound, and closing the incision with a staple or a suture, at least a portion of the staple or suture formed over the pressure relief device and positioned in the feature. The positioning step may further include, for example, positioning the pressure relief device alongside the incision. Alternatively, the positioning step may further include, for example, positioning the pressure relief device across the incision. The closing step may include forming a simple interrupted suture, a horizontal mattress suture, or a vertical mattress suture. The closing step may further include forming a knot of the simple interrupted suture, the horizontal mattress suture, or the vertical mattress suture on a top surface of the body. The closing step may include using a staple and a suture. In some embodiments, multiple pressure relief devices 1300 are used to close the incision.

The shape of the body of the pressure relief device 1300 may be varied to accommodate various suture patterns, suture sizes, staple patterns, staple sizes, and or incision lengths. Several example embodiments will now be described.

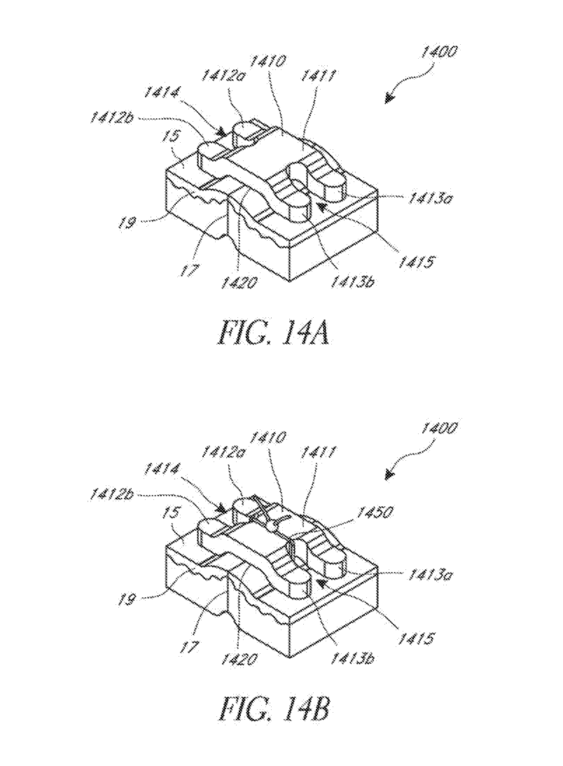

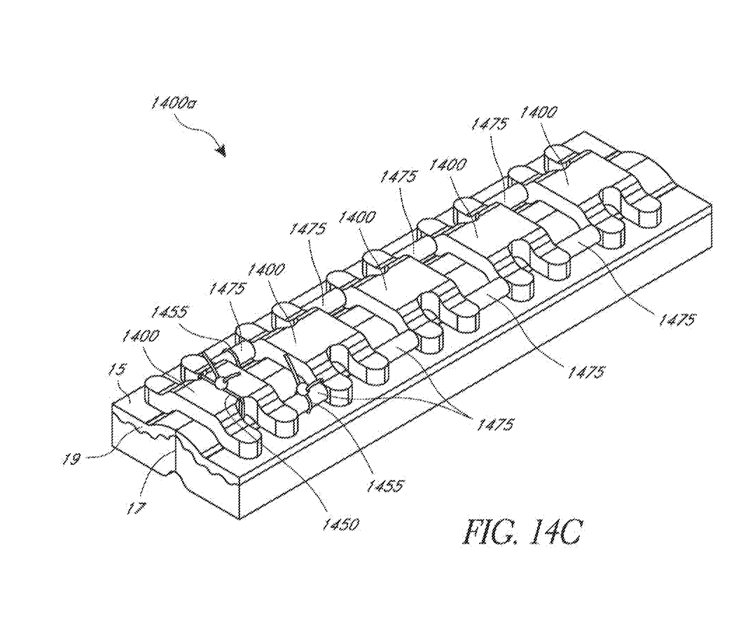

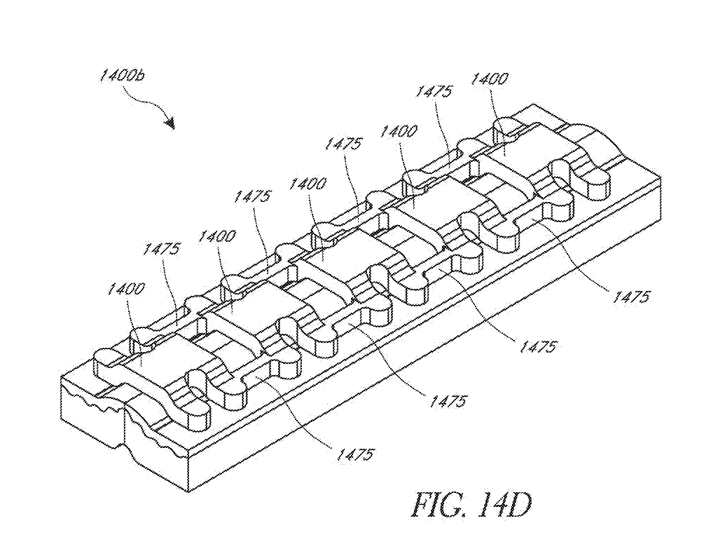

FIG. 14A shows a perspective view of one embodiment of a pressure relief device 1400. The device 1400 includes a body 1410. The body 1410 may be configured for use with a simple interrupted suture. In the illustrated embodiment, the body 1410 is substantially H-shaped. The body includes a central portion 1411. On a first side, two legs 1412a, 1412b extend away from the central portion 1411. A slot 1414 is formed between the two legs 1412a, 1412b. The two legs 1412a, 1412b may be substantially parallel with each other. When the device 1400 is positioned on the skin 15 for use, the two legs 1412a, 1412b may extend substantially orthogonally to the incision 17. On a second side, two legs 1413a, 1413b extend away from the central portion 1411. A slot 1415 is formed between the two legs 1413a, 1413b. The two legs 1413a, 1412b may be substantially parallel with each other. When the device 1400 is positioned on the skin 15 for use, the two legs 1413a, 1413b may extend substantially orthogonally to the incision 17. The body 1410 may be shaped to include an eversion recess 1420 in the bottom surface thereof. The eversion recess 1420 accommodates a region of wound eversion 19.

FIG. 14B shows the pressure relief device 1400 of FIG. 14A in use with a simple interrupted suture 1450. As shown, the simple interrupted suture 1450 extends over the central portion 1411 of the body 1410. An external portion of the suture 1450 is positioned above and rests on the body 1410. The suture 1450 may be secured with a knot. The knot may rest on the body 1410. The suture 1450 enters the skin 1450 in the slots 1414, 1415 on each side of the incision 17. The depth of the slots 1414, 1415 and the width of the central portion 1411 may be configured to match the width of the suture stitch. The device 1400 may also be used with a staple. For example, a crown of the staple may extend across the central portion 1411. Legs of the staple extend down through the slots 1414, 1415 into the skin 1415.

FIG. 14C shows an embodiment of a pressure relief device 1400a configured for use with multiple sutures. The pressure relief device 1400a includes a plurality of pressure relief devices 1400 (for example, as shown in FIGS. 14A-14B) connected to form a strip. In the illustrated embodiment, five pressure relief devices 1400 are connected, but greater or fewer numbers may be used. The pressure relief devices 1400 are connected by bridges 1475 extending between each pressure relief device 1400. In the illustrated embodiment, the bridges 1475 include cylinders. In the illustrated embodiment, two bridges 1475 extend between each pair of pressure relief devices, one on each side of the incision 17. The length of each bridge 1475 may be configured to control the spacing between sutures, and accordingly, the pressure relief device 1400a provides a template for where to place stitches.

As shown, a simple interrupted suture 1450 may be formed over a pressure relief device 1400 as described above. The bridges 1475 may be configured for use with a vertical mattress suture 1455, as shown. A first external portion of the vertical mattress suture 1455 is formed over one bridge 1475 and a second external portion of the vertical mattress suture 1455 is formed over the other bridge 1475.

FIG. 14D shows another embodiment of a pressure relief device 1400a configured for use with multiple sutures. The pressure relief device 1400b is similar to pressure relief device 1400a described above, except the bridges include rectangular prisms. The pressure relief device 1400b may be used with simple interrupted sutures and/or vertical mattress sutures. In further embodiments, the bridges may include other shapes.

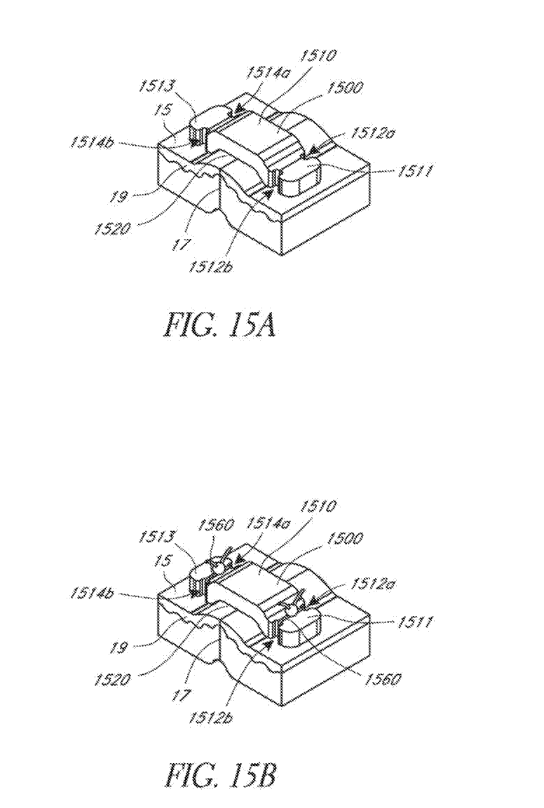

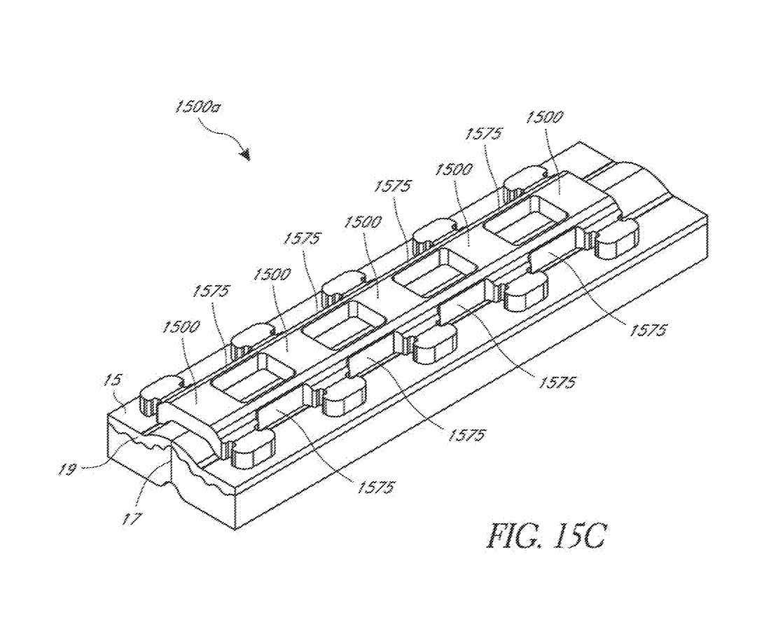

FIG. 15A shows a perspective view of another embodiment of a pressure relief device 1500. The device 1500 includes a body 1510. The body 1510 may be configured for use with a horizontal mattress suture, for example. In the illustrated embodiment, the body 1510 is substantially rectangular. On a first side of the body 1510, a T-shaped flange 1511 extends away from the body 1510. The T-shaped flange 1511 includes two opposing grooves 1512a, 1512b that extend through the body flange 1511 from the top surface to the bottom surface. On a second side of the body 1510, a T-shaped flange 1513 extends away from the body 1510. The T-shaped flange 1513 includes two opposing grooves 1514a, 1514b that extend through the body flange 1511 from the top surface to the bottom surface. In use, the device 1500 may be positioned such that the T-shaped flanges 1511, 1513 are positioned on opposite sides of an incision 17. The body 1510 may be shaped to include an eversion recess 1520 in the bottom surface thereof. The eversion recess 1520 accommodates a region of wound eversion 19.

FIG. 15B shows the pressure relief device 1500 of FIG. 15A in use with a horizontal mattress suture 1560. A first external portion of the suture 1560 is formed over the T-shaped flange 1511 and a second external portion of the suture 1560 is formed over the T-shaped flange 1513. The suture 1560 passes through the grooves 1512a, 1512b, 1514a, 1514b to enter the skin. The suture 1560 may be secured with a knot. The knot may rest on one of the T-shaped flanges 1511, 1512. The device 1500 may also be used with a simple interrupted suture or staple formed over each of the T-shaped flanges 1511, 1512.

FIG. 15C shows another embodiment of a pressure relief device configured for use with multiple sutures. The pressure relief device 1500a includes a plurality of pressure relief devices 1500 (for example, as shown in FIGS. 15A-15B) connected to form a strip. In the illustrated embodiment, five pressure relief devices 1500 are connected, but greater or fewer numbers may be used. The pressure relief devices 1500 are connected by bridges 1575 extending between each pressure relief device 1500. In the illustrated embodiment, the bridges 1575 include rectangular prisms, although other shapes may be used. In the illustrated embodiment, two bridges 1575 extend between each pair of pressure relief devices 1500, one on each side of the incision 17. The length of each bridge 1575 may be configured to control the spacing between sutures, and accordingly, the pressure relief device 1400a provides a template for where to place stitches. In some embodiments, vertical mattress sutures or staples may be formed over the bridges 1575.

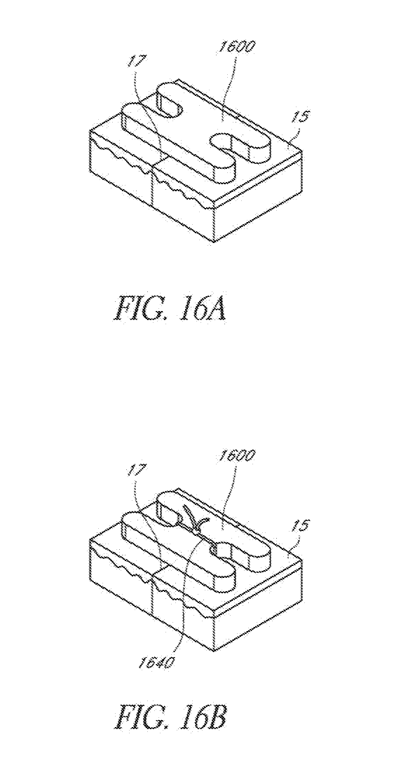

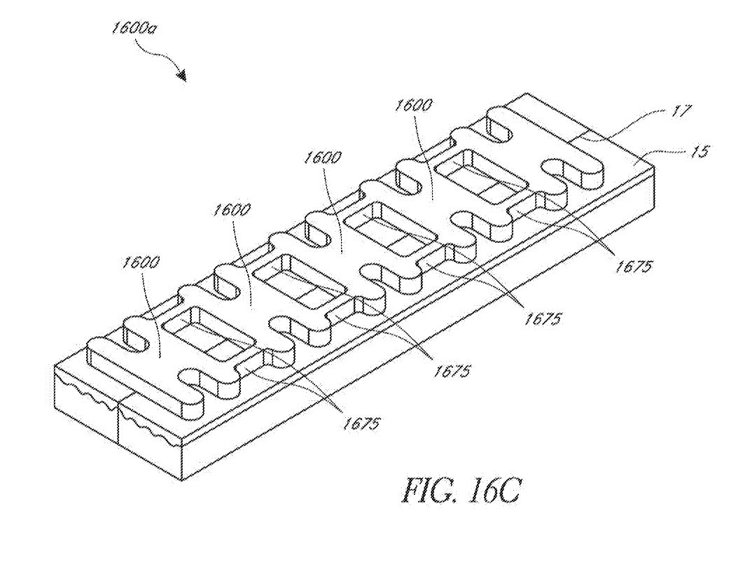

FIG. 16A shows a perspective view of another embodiment of a pressure relief device 1600. The pressure relief device 1600 may be substantially similar to the pressure relief device 1400 described above, except the device 1600 is substantially flat. The device 1600 may not include an eversion recess. FIG. 16B shows the pressure relief device of FIG. 16A in use with a simple interrupted suture 1640. FIG. 16C shows another embodiment of a pressure relief device 1600a configured for use with multiple sutures and/or staples. The pressure relief device 1600a may be substantially similar to the pressure relief device 1400a described above, except the device 1600a is substantially flat. The device 1600a may not include an eversion recess.

The embodiments of pressure relief devices shown in the figures and described in the text are provided by way of example only. The features of these embodiments may be altered, modified, combined, etc. in a variety of ways which will be apparent to one of skill in the art upon review and implementation of this disclosure. For example, any of the devices may be modified for use with staples and/or sutures of varying types.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. Applicant reserves the right to submit claims directed to combinations and sub-combinations of the disclosed inventions that are believed to be novel and non-obvious. Inventions embodied in other combinations and sub-combinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. A staple, comprising:
a crown extending substantially along an axis between a first end and a second end;
a first leg extending from the first end of the crown, the first leg ending in a point;
a second leg extending from the second end of the crown, the second leg ending in a point;
the first leg and the second leg deformably moveable between a first configuration and a second configuration, in the first configuration the first leg and the second leg are positioned substantially parallel with the crown, in the second configuration the first leg and the second leg positioned are substantially perpendicular to the crown; and
a pad disposed on at least a portion of the crown.

2. The staple of claim 1, wherein the pad surrounds at least a portion of the crown.

3. The staple of claim 1, wherein the pad is positioned substantially at the midpoint of the crown between the first end and the second end.

4. The staple of claim 1, wherein the pad comprises a substantially cylindroid shape body.

5. The staple of claim 1, wherein the pad comprises a substantially rectangular body, and wherein the crown extends through the rectangular body from a first side of the rectangular body to a second side of a rectangular body.

6. The staple of any claim 5, wherein each of the first side and the second side of the rectangular body include a groove formed therein, the groove extending along an axis substantially perpendicular to the crown.

7. The staple of claim 5, wherein a top surface of the rectangular body includes a notch formed therein, the notch extending along an axis substantially perpendicular to the crown.

8. The staple of claim 5, wherein a bottom surface of the rectangular body includes an eversion recess formed therein, the eversion recess extending through the rectangular body along an axis substantially perpendicular to the crown.

9. The staple of claim 8, further comprising a foot positioned on each side of the eversion recess.

10. The staple of claim 1, wherein the pad comprises one or more soft synthetic polymers of type A shore durometer of 100 or less.

11. The staple of claim 1, wherein the pad comprises one or more hard synthetic polymers of type D shore durometer of 0 or more.

12. The staple of claim 1, wherein the pad comprises a first pad and the staple further comprises a second pad surrounding at least another portion of the crown, the first pad separated from the second pad.

13. The staple of claim 12, wherein the first pad and the second pad each comprises a substantially cylindroid shape.

14. The staple of claim 12, wherein the first pad and the second pad each comprises silicone overmolded on the crown.

15. The staple of claim 1, wherein each of the first leg and the second leg comprise:
an extension portion extending from the crown; and
a tooth extending from the end of the extension portion to a distal end, the point located at the distal end of the tooth.

16. The staple of claim 15, wherein the tooth extends substantially orthogonally from the extension portion.

17. The staple of claim 16, wherein the tooth is curved.

18. The staple of claim 1, wherein the pad is configured to elevate the crown above a surface of a patient's skin when the staple is in use.

19. A staple, comprising:
a crown extending between a first end and a second end;
a first leg extending from the first end of the crown, the first leg ending in a point;
a second leg extending from the second end of the crown, the second leg ending in a point;
the first leg and second leg deformably moveable between a first configuration and a second configuration, in the first configuration the first leg and the second leg are positioned substantially parallel with the crown, in the second configuration the first leg and the second leg are positioned substantially perpendicular to the crown; and
the crown shaped such that at least a portion of the crown is elevated above a surface of a patient's skin when the staple is in use.

20. The staple of claim 19, wherein the crown further comprises a central portion located between the first end and the second end, the central portion comprising a U-shape.

21. The staple of claim 20, wherein the U-shape extends in the same direction as the first leg and the second leg in the second configuration.

22. The staple of claim 20, wherein the U-shape extends in the opposite direction as the first leg and the second leg in the second configuration.

23. The staple of claim 20, further comprising a pad disposed on a bottom portion of the U-shape.

24. The staple of claim 23, wherein the pad surrounds at least a portion of the crown.

25. The staple of claim 23, wherein the pad comprises a substantially cylindroid shape.

26. The staple of claim 23 24, wherein the pad comprises an H-shape, and wherein the central portion of the U-shape extends through a central portion of the H-Shape, in the first configuration lateral portions of the H-shape are substantially parallel with the first leg and the second leg, and in the second configuration the first leg and the second leg are perpendicular to and extend between the lateral portions of the H-shape.

27. The staple of claim 23, wherein the pad comprises silicone overmolded on the crown.

28. The staple of claim 19, wherein the crown comprises:
a first U-shape adjacent to the first end;
a second U-shape adjacent to the second end, the first U-shape and the second U-shape located in a single plane; and
a central portion located between the first U-shape and the second U-shape.

29. The staple of claim 28, further comprising a pad disposed on at least a portion of the central portion.

30. The staple of claim 29, wherein the pad surrounds at least a portion of the crown.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)
